(19) United States
(12) Patent Application Publication
(10) Pub. No.: US 2024/0382279 A1
(43) Pub. Date: Nov. 21, 2024

(54) SURGICAL INSTRUMENT CABLE CONTROL AND ROUTING STRUCTURES

(71) Applicant: Intuitive Surgical Operations, Inc., Sunnyvale, CA (US)

(72) Inventors: Michael BALDWIN, Los Gatos, CA (US); Erik NELSON, Durango, CO (US); Matthew A. WIXEY, Rochester, CT (US)

(73) Assignee: Intuitive Surgical Operations, Inc., Sunnyvale, CA (US)

(21) Appl. No.: 18/683,651

(22) PCT Filed: Aug. 10, 2022
(86) PCT No.: PCT/US2022/039942

§ 371 Date: Feb. 14, 2024

Related U.S. Application Data
(60) Provisional application No. 63/233,904, filed on Aug. 17, 2021.

(51) Int. Cl. A61B 34/00; A61B 17/00

(52) U.S. Cl. CPC A61B 34/71; A61B 2017/00323

(57) ABSTRACT

A medical device includes a chassis component having a bottom and an opening defined in the bottom, and a capstan including an upper portion, a lower portion, and a spool between the upper and lower portions. The upper portion of the capstan is supported within the opening of the chassis component. The spool comprises a cable wrap surface and a side wall opposing the bottom of the upper chassis. The side wall of the spool can optionally slope away from the bottom of the chassis component. The medical device can further include a cable guide and a cable. The cable extends from the cable guide to the cable wrap surface of the spool.

### CROSS-REFERENCE TO RELATED APPLICATIONS

This application claims priority to and the benefit of U.S. Provisional Patent Application No. 63/233,904, entitled “Surgical Instrument Cable Control and Routing Structures,” filed Aug. 17, 2021, the disclosure of which is incorporated herein by reference in its entirety.

### BACKGROUND

The embodiments described herein relate to medical devices, and more specifically to endoscopic tools. More particularly, the embodiments described herein relate to devices that include capstans, tension cables, and mechanisms for coupling the cables within a medical device.

Known techniques for Minimally Invasive Surgery (MIS) employ instruments to manipulate tissue that can be either manually controlled or controlled via computer-assisted teleoperation. Many known MIS instruments include a therapeutic or diagnostic end effector (e.g., forceps, a cutting tool, or a cauterizing tool) mounted on a wrist mechanism at the distal end of a shaft. During an MIS procedure, the end effector, wrist mechanism, and the distal end of the shaft are inserted into a small incision or a natural orifice of a patient to position the end effector at a work site within the patient's body. The optional wrist mechanism can be used to change the end effector's orientation with reference to the shaft to perform the desired procedure at the work site. Known wrist mechanisms generally provide the desired mechanical degrees of freedom (DOFs) for movement of the end effector. For example, known wrist mechanisms are able to change the pitch and yaw orientation of the end effector with reference to the shaft's longitudinal axis. A wrist may optionally provide a roll DOF for the end effector with reference to the shaft, or an end effector roll DOF may be implemented by rolling the shaft, wrist, and end effector together as a unit. An end effector may optionally have additional mechanical DOFs, such as grip or knife blade motion. In some instances, wrist and end effector mechanical DOFs may be combined to provide various end effector control DOFs. For example, U.S. Pat. No. 5,792,135 (filed May 16, 1997) discloses a mechanism in which wrist and end effector grip mechanical DOFs are combined to provide an end effector yaw control DOF.

To enable the desired movement of the distal wrist mechanism and end effector, known instruments include cables that extend through the shaft of the instrument and that connect the wrist mechanism to a mechanical structure configured to move the cables to operate the wrist mechanism and end effector. For teleoperated systems, the mechanical structure is typically motor driven and is operably coupled to a computer processing system to provide a user interface for a clinical user (e.g., a surgeon) to control the instrument as a whole, as well as the instrument's components and functions.

Patients benefit from continual efforts to improve the effectiveness of MIS methods and devices. For example, reducing the size and/or the operating footprint of the shaft and wrist mechanism can allow for smaller entry incisions and reduced need for space at the surgical site, thereby reducing the negative effects of surgery, such as pain, scarring, and undesirable healing time. But producing small medical devices that implement the clinically desired functions for minimally invasive procedures can be challenging. Specifically, simply reducing the size of known wrist mechanisms by scaling down the components will not result in an effective solution because required component and material properties do not scale at relatively small physical dimensions. For example, efficient implementation of a wrist mechanism can be complicated because the cables must be carefully routed through the wrist mechanism to maintain cable tension throughout the range of motion of the wrist mechanism or end effector and to minimize the interactions (coupling effects) of motion about one rotation axis upon motion about another rotation axis. As another example, pulleys and/or contoured surfaces are generally needed to reduce cable friction, which extends instrument life and permits operation without excessive forces being applied to the cables or other structures in the wrist mechanism. But increased localized forces that may result from smaller structures and cable bend radii (including smaller diameter cables and other wrist and end effector components) can result in undesirable lengthening (e.g., stretch or creep) of the cables during storage and use, reduced cable life, and the like.

Further, the wrist mechanism generally provides specific degrees of freedom for movement of the end effector. For example, for forceps or other grasping tools, the wrist may be able to change the end effector pitch, yaw, and grip orientations with reference to the instrument shaft. More degrees of freedom could be implemented through the wrist but would require additional actuation members (e.g., cables) in the wrist and shaft, and these additional members compete for the limited space that exists given the size restrictions required by MIS applications. Components needed to actuate other degrees of freedom, such as end effector roll or insertion/withdrawal through movement of the main tube, also compete for space at or in the shaft of the device.

A conventional architecture for a wrist mechanism in a manipulator-driven medical device uses cables pulled in and payed out by a capstan in the proximal mechanical structure and thereby rotate the portion of the wrist mechanism that is connected to the capstan via the cables. For example, a wrist mechanism can be operably coupled to three capstans-one each for rotations about a pitch axis, a yaw axis, and a grip axis. Each capstan can be controlled by using two cables that are attached to the capstan so that one side pays out cable while the other side pulls in an equal length of cable. With this architecture, three degrees of freedom require a total of six cables extending from the wrist mechanism proximally back along the length of the instrument's main shaft tube to the instrument's proximal mechanical structure. Efficient implementation of a wrist mechanism and proximal mechanical structure can be complicated because the cables must be carefully routed through the tool member, wrist mechanism, and proximal mechanical structure to maintain stability of the wrist throughout the range of motion of the wrist mechanism and to minimize the interactions (or coupling effects) of one rotation axis upon another.

Some known architectures for a manipulator-driven medical device use cables including crimps or other retention methods to secure the cables to the capstan or to the tool member, which can increase the time and costs of manufacturing the medical device. For example, there may be increased time needed for routing and securing the crimps to the capstan and/or end effector. In addition, the cables themselves can be very expensive. For example, many conventional architectures for manipulator-driven medical devices use cables made from materials such as, tungsten or steel. Such cables can be constructed for extended use over time (e.g., used during several separate surgical procedures on different patients) but are also very expensive.

In some known manipulator-driven medical device architectures that use cables and that turn a capstan in the proximal mechanical structure and thereby rotate the portion of the wrist mechanism that is connected to the capstan, slack in the untensioned cables can result when the instrument is in an uninstalled (a “pre-operational state”) state, such as during storage, prior to use on an associated surgical system, or prior to full engagement for use when installed on an associated surgical system.

To establish an effective control relationship between capstan rotation and desired wrist or end effector motion in an operational state, slack in the cables connecting the capstan and the wrist or end effector must be eliminated. But eliminating such slack within a medical device context is challenged by several design constraints. First, a significant length of cable slack may develop so that cable tangling or fouling must be prevented as the slack is taken up on a capstan. An effective way of guiding the slack cable onto the capstan without tangling or fouling is needed. Second, cable slack may snag on a structural element within the instrument as the cable slack is taken up. The volume in which the cable slack exists within the medical device must be clear of potentially snagging elements. Third, instrument size must be kept small to comply with telesurgical system requirements. Even if a capstan with a large takeup spool with opposing side walls is used to prevent cable tangling, such a capstan may be too large and long for effective use. An effective design to allow cable slack takeup within a medical device size constraints is required. Fourth, material and manufacturing costs should be kept low in order to keep health care costs as low as possible. Fifth, an effective cable slack takeup design is needed for some optional capstan configurations to allow effective coupling of the cable to the capstan. These design constraints together, as well as other medical device design requirements, provide a multi-faceted challenge.

Thus, a need exists for improved medical devices, including improved proximal mechanical structures for such medical devices to enable a wrist to be operated, that provides a mechanical structure that eliminates the need for complex mechanisms to guide the cable when moved from an untensioned, pre-operational configuration to an operational configuration.

A need also exists for improved medical devices, including improved proximal mechanical structures to enable a wrist to be operated with a small number of cables, to facilitate miniaturization of the device and to reduce manufacturing cost by reducing the number of parts required.

### SUMMARY

This summary introduces certain aspects of the embodiments described herein to provide a basic understanding. This summary is not an extensive overview of the inventive subject matter, and it is not intended to identify key or critical elements or to delineate the scope of the inventive subject matter.

In some embodiments, a medical device, includes a chassis component having a bottom and an opening defined in the bottom, and a capstan including an upper portion, a lower portion, and a spool between the upper and lower portions. The upper portion of the capstan is supported within the opening of the chassis component. The spool includes a cable wrap surface and a side wall opposing the bottom of the upper chassis. The side wall of the spool optionally slopes away from the bottom of the chassis component.

In some embodiments, the medical device further includes a cable guide and a cable. The cable extends from the cable guide to the cable wrap surface of the spool. In some embodiments, the cable is coupled to the spool such that the cable is routed about the cable wrap surface of the spool by no more than two revolutions.

In some embodiments, on a condition in which the cable is in an untensioned state, a slack loop exists in the cable between the cable guide and the spool. In some embodiments of the medical device, on a condition in which the capstan rotates to wind the cable about the cable wrap surface of the spool to cause the cable to transition from the untensioned state to a tensioned state, the slack loop of the cable is guided onto the spool by the bottom of the chassis component and the side wall of the spool.

In some embodiments, the medical device further includes a tool member that moves from a first motion limit position through a defined range of motion to a second motion limit position. The cable is coupled to the spool such that the cable is routed about the cable wrap surface of the spool by no more than two revolutions as the spool rotates to move the tool member through the defined range of motion. In some embodiments, the side wall of the spool has an outer circumference and a radiused edge at the outer circumference.

In some embodiments, the medical device further includes a cable and a bearing. The cable includes a proximal portion coupled to the capstan and the bearing supports the upper portion of the capstan within the chassis component and surrounds the proximal portion of the cable. In some embodiments, the proximal portion of the cable is coupled to the capstan in a wrapped configuration and the bearing surrounds the proximal portion of the cable to assist in maintaining the wrapped configuration of the proximal portion of the cable.

In some embodiments, the bearing is a first bearing and the medical device further includes a second bearing. The first bearing is coupled to the upper portion of the capstan and the second bearing supports the lower portion of the capstan. In some embodiments, the first bearing is a rolling-element bearing and the second bearing is a journal bearing. In some embodiments, the spool of the capstan is located adjacent the chassis component such that on a condition in which tension in the cable causes a lateral load on the capstan, a portion of the lateral load on the first bearing is larger than a portion of the lateral load on the second bearing.

In some embodiments, the cable is a polymeric braided construction. In some embodiments, the spool includes a second side wall opposite the side wall and the second side wall of the spool is located within the opening of the chassis component. In some embodiments, a diameter of the capstan tapers inward from the spool toward the bottom portion of the capstan. In some embodiments, the capstan includes a plurality of radial ribs between the spool and the lower portion of the capstan and a diameter of each rib from the plurality of radial ribs decreases between the spool and the lower portion of the capstan. In some embodiments, the capstan consists essentially of a monolithic polymer material. In some embodiments, the upper portion of the capstan includes a cable anchor portion and a proximal portion of the cable is secured to the capstan with a wrapping about the cable anchor portion.

In some embodiments, a medical device includes a shaft including a distal end portion and a proximal end portion, a tool member coupled to the distal end portion of the shaft and a mechanical structure coupled to the proximal end portion of the shaft. The mechanical structure includes a cable guide, chassis component, and a capstan. The capstan includes a first portion, a second portion, and a drive surface between the first portion and the second portion. The chassis component has a bottom and a cable coupled to the tool member is routed through the cable guide, and about the drive surface of the capstan. The cable includes a slack loop between the drive surface of the capstan and the cable guide when the cable is in an untensioned state. When the cable transitions from the untensioned state to a tensioned state, the slack loop of the cable is guided onto the capstan by the bottom of the chassis component and the second portion of the capstan.

In some embodiments, the cable is routed about the drive surface of the capstan by no more than two revolutions. In some embodiments, the second portion of the capstan has a radiused top edge surface. In some embodiments, the mechanical structure includes a bearing. the cable includes a proximal portion coupled to the first portion of the capstan, and the bearing is coupled to the capstan and surrounds the proximal portion of the cable to assist in maintaining the proximal portion of the cable coupled to the first portion of the capstan.

In some embodiments, the bearing is a first bearing and the medical device includes a second bearing. The first bearing is coupled to the first portion of the capstan and the second bearing is coupled to the second portion of the capstan. In some embodiments, the cable is a polymeric braided construction.

In some embodiments, a medical device includes an instrument shaft, a cable guide, a capstan, and a cable. The instrument shaft includes a shaft passageway and an opening is defined through the cable guide. The cable guide includes two opposing protrusions, and the protrusions define a guide slot in communication with the opening of the cable guide. The cable has a first width and the guide slot has a second width smaller than the first width of the cable. The cable is routed from the capstan, through the opening of the cable guide, and into the shaft passageway. In some embodiments, on a condition in which the cable is in an untensioned state, the protrusions of the cable guide maintain the cable within the opening of the cable guide.

In some embodiments, the cable is coupled to the capstan such that the cable has a slack loop between the capstan and the cable guide when the cable is in the untensioned state. In some embodiments, the medical device includes a chassis component having a bottom and the capstan includes a first portion, a second portion, and a drive surface. When the cable transitions from the untensioned state to a tensioned state, the slack loop of the cable is guided onto the capstan by the bottom of the chassis component and the second portion of the capstan. In some embodiments, the capstan includes a first portion, a second portion, and a drive surface. The cable is coupled to the capstan such that the cable is routed about the drive surface of the capstan by no more than two revolutions.

Other medical devices, related components, medical device systems, and/or methods according to embodiments will be or become apparent to one with skill in the art upon review of the following drawings and detailed description. It is intended that all such additional medical devices, related components, medical device systems, and/or methods included within this description be within the scope of this disclosure.

### BRIEF DESCRIPTION OF THE DRAWINGS

FIG. 1 is a plan view of a minimally invasive teleoperated medical system according to an embodiment being used to perform a medical procedure such as surgery.

FIG. 2 is a perspective view of an optional auxiliary unit of the minimally invasive teleoperated surgery system shown in FIG. 1.

FIG. 3 is a perspective view of a user control console of the minimally invasive teleoperated surgery system shown in FIG. 1.

FIG. 4 is a front view of a manipulator unit, including a plurality of instruments, of the minimally invasive teleoperated surgery system shown in FIG. 1.

FIG. 5A is a diagrammatic illustration of a portion of a medical device according to an embodiment, shown in a tensioned configuration.

FIG. 5B is a diagrammatic illustration of a portion of the medical device of FIG. 5A, shown in an untensioned configuration.

FIG. 6A is a diagrammatic illustration of a portion of the mechanical structure of the medical device of FIG. 5A.

FIG. 6B is a diagrammatic illustration of a portion of the mechanical structure of the medical device of FIG. 5A showing a cable guide and cable coupled to the capstan and cable guide.

FIG. 7A is a structural illustration of a portion of a medical device, according to another embodiment, illustrating a cable extending between a capstan and an end effector.

FIG. 7B is a structural illustration of a portion of the medical device of FIG. 7A illustrating cable travel during grip movements of the end effector.

FIG. 7C is a structural illustration of a portion of the medical device of FIG. 7A illustrating cable travel during yaw movements of the end effector.

FIG. 7D is a structural illustration of a portion of the medical device of FIG. 7A illustrating cable travel during pitch movements of the end effector.

FIG. 8 is a perspective view of a medical device according to an embodiment.

FIG. 9 is a perspective view of the distal end portion of the medical device of FIG. 8 with an outer cover removed and the tool member in a closed position.

FIG. 10 is a top view of the distal end portion of the medical device of FIG. 8 with the outer cover removed and the tool member in the closed position.

FIG. 11A is a side perspective view of a portion of the mechanical structure of the medical device of FIG. 8 with select components removed for illustration purposes.

FIG. 11B is a distal end perspective view of the portion of the mechanical structure shown in FIG. 11A.

FIG. 11C is a side perspective view of a portion of the mechanical structure of the medical device of FIG. 8 with select components removed for illustration purposes.

FIG. 11D is a side perspective view of a portion of the mechanical structure of the medical device of FIG. 8 with select components removed for illustration purposes.

FIG. 12A is a front view of a capstan of the medical device of FIG. 8.

FIG. 12B is a rear view of the capstan of FIG. 12A.

FIG. 13A is a side view of a drive disc of the medical device of FIG. 8.

FIG. 13B is a bottom perspective view of the drive disc of FIG. 13A.

FIG. 14 is a top perspective view of a portion of the medical device of FIG. 8 illustrating cables extending between the capstans and a cable guide.

FIG. 15A is a side perspective view of a cable guide of the medical device of FIG. 8.

FIG. 15B is an enlarged top view of a portion of the cable guide of the medical device of FIG. 8, illustrating cables routed through the cable guide.

FIG. 16A is an enlarged diagrammatic illustration of a top portion of the cable guide of FIGS. 15A and 15B.

FIGS. 16B-16D are each a diagrammatic illustration of a portion of the cable guide of the medical device of FIG. 8, showing the insertion of a cable through a slot of the cable guide.

FIG. 17 is a side view of a portion of the mechanical structure of the medical device of FIG. 8 illustrating a cable in an untensioned state.

FIG. 18 is a perspective view of a portion of a capstan of a medical device according to an embodiment.

FIG. 19 is a front view of the portion of the capstan of FIG. 18.

FIG. 20 is a back view of the portion of the capstan of FIG. 18.

FIG. 21 is a top view of the portion of the capstan of FIG. 18.

FIG. 22 is a bottom view of the portion of the capstan of FIG. 18.

FIGS. 23-29 each illustrate a step in a wrap sequence for a cable to be coupled to the portion of the capstan of FIG. 18.

FIG. 30 is a side view of a portion of a cable, according to an embodiment.

### DETAILED DESCRIPTION

The embodiments described herein can advantageously be used in a wide variety of grasping, cutting, and manipulating operations associated with minimally invasive surgery. In some embodiments, an end effector of the medical device can move with reference to the main body of the instrument in three mechanical DOFs, e.g., pitch, yaw, and roll (shaft roll). There may also be one or more mechanical DOFs in the end effector itself, e.g., two jaws, each rotating with reference to a clevis (2 DOFs) and a distal clevis that rotates with reference to a proximal clevis (one DOF).

The medical devices of the present application enable motion in three degrees of freedom (e.g., about a pitch axis, a yaw axis, and a grip axis) using only four cables, thereby reducing the total number of cables required, reducing the space required within the shaft and wrist, reducing overall cost, and enables further miniaturization of the wrist and shaft assemblies to promote MIS procedures. Moreover, the instruments described herein include one or more cables (which function as tension members) that are made of a polymer material and that can be secured to a capstan of the proximal end mechanism without the need for a retention element or other securing feature. In some embodiments, the capstans are configured with grooves, and a cable is wrapped about a capstan and disposed at least partially within the grooves such that a first wrap portion of the cable crosses over a second wrap portion of the cable. The cross-over configuration assists in securing the cables to the capstans. The polymer material of the cable or a coating applied to the surface of the cable also provides sufficient friction to further assist in maintaining the cable secured to the capstan without the need for any additional mechanical features for securing the cable to the capstan (e.g., placing cable crimps within a guide slot, securing the cable to the capstan with an adhesive, or the like).

Additionally, the instruments described herein have an architecture that provides for better control and guidance of the cables during operation by accommodating a slack loop portion of the cable that is present when the instrument and cables are in an untensioned state (e.g., during storage). More specifically, the mechanical structure provides a guide path for the cables between the capstans, to which the cables are coupled within the mechanical structure, and a bottom of a chassis component of the mechanical structure. When the cables are moved to a tensioned state (e.g., operational state), the slack loop portion is guided within these structures to remain within a desired working path of the medical device. Thus, rather than using mechanisms to eliminate or reduce the slack in the cable (e.g., a movable tensioner pulley, a spring-loaded guide surface that moves to maintain cable tension, or a ratchet mechanism to maintain cable tension), the slack loop portion of the cable can be controlled and guided as the cable is tensioned. Controlling the slack loop portion can be particularly beneficial when using cables formed with, for example, a polymer material, which can increase the potential for and amount of slack loop formed within the cable in the untensioned state.

As used herein, the term “about” when used in connection with a referenced numeric indication means the referenced numeric indication plus or minus up to 10 percent of that referenced numeric indication. For example, the language “about 50” covers the range of 45 to 55. Similarly, the language “about 5” covers the range of 4.5 to 5.5.

As used in this specification and the appended claims, the word “distal” refers to direction towards a work site, and the word “proximal” refers to a direction away from the work site. Thus, for example, the end of a medical device that is closest to the target tissue would be the distal end of the medical device, and the end opposite the distal end (i.e., the end manipulated by the user or coupled to the actuation shaft) would be the proximal end of the medical device.

Further, specific words chosen to describe one or more embodiments and optional elements or features are not intended to limit the invention. For example, spatially relative terms-such as “beneath”, “below”, “lower”, “above”, “upper”, “proximal”, “distal”, and the like-may be used to describe the relationship of one element or feature to another element or feature as illustrated in the figures. These spatially relative terms are intended to encompass different positions (i.e., translational placements) and orientations (i.e., rotational placements) of a device in use or operation in addition to the position and orientation shown in the figures. For example, if a device in the figures is turned over, elements described as “below” or “beneath” other elements or features would then be “above” or “over” the other elements or features. Thus, the term “below” can encompass both positions and orientations of above and below. A device may be otherwise oriented (e.g., rotated 90 degrees or at other orientations) and the spatially relative descriptors used herein interpreted accordingly. Likewise, descriptions of movement along (translation) and around (rotation) various axes includes various spatial positions and orientations. The combination of a body's position and orientation define the body's pose.

Similarly, geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

In addition, the singular forms “a”, “an”, and “the” are intended to include the plural forms as well, unless the context indicates otherwise. The terms “comprises”, “includes”, “has”, and the like specify the presence of stated features, steps, operations, elements, components, etc. but do not preclude the presence or addition of one or more other features, steps, operations, elements, components, or groups.

Unless indicated otherwise, the terms apparatus, medical device, medical instrument, and variants thereof, can be interchangeably used.

Aspects of the invention are described primarily in terms of an implementation using a da Vinci® surgical system, commercialized by Intuitive Surgical, Inc. of Sunnyvale, California. Examples of such surgical systems are the da Vinci Xi® surgical system (Model IS4000), da Vinci X® Surgical System (Model IS4200), and the da Vinci Si® surgical system (Model IS3000). Knowledgeable persons will understand, however, that inventive aspects disclosed herein may be embodied and implemented in various ways, including computer-assisted, non-computer-assisted, and hybrid combinations of manual and computer-assisted embodiments and implementations. Implementations on da Vinci® surgical systems (e.g., the Model IS4000, the Model IS3000, the Model IS2000, the Model IS1200, the Model SP1099) are merely presented as examples, and they are not to be considered as limiting the scope of the inventive aspects disclosed herein. As applicable, inventive aspects may be embodied and implemented in both relatively smaller, hand-held, hand-operated devices that are not mechanically grounded in a world reference frame and relatively larger systems that have additional mechanical support that is grounded in a world reference frame.

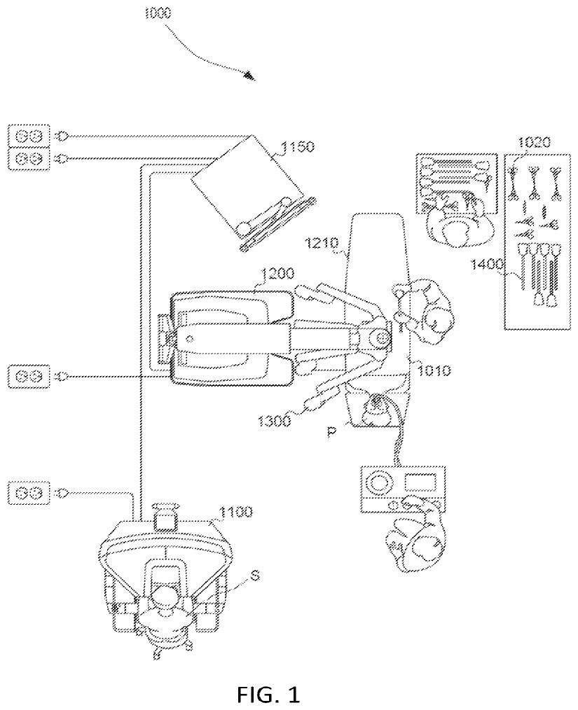

FIG. 1 is a plan view illustration of a teleoperated surgical system 1000 that operates with at least partial computer assistance (a “telesurgical system”). Both telesurgical system 1000 and its components are considered medical devices. Telesurgical system 1000 is a Minimally Invasive Robotic Surgical (MIRS) system used for performing a minimally invasive diagnostic or surgical procedure on a Patient P who is lying on an Operating table 1010. The system can have any number of components, such as a user control unit 1100 for use by a surgeon or other skilled clinician S during the procedure. The MIRS system 1000 can further include a manipulator unit 1200 (popularly referred to as a surgical robot) and an optional auxiliary equipment unit 1150. The manipulator unit 1200 can include an arm assembly 1300 and a surgical instrument tool assembly removably coupled to the arm assembly. The manipulator unit 1200 can manipulate at least one removably coupled instrument 1400 through a minimally invasive incision in the body or natural orifice of the patient P while the surgeon S views the surgical site and controls movement of the instrument 1400 through control unit 1100. An image of the surgical site is obtained by an endoscope (not shown), such as a stereoscopic endoscope, which can be manipulated by the manipulator unit 1200 to orient the endoscope. The auxiliary equipment unit 1150 can be used to process the images of the surgical site for subsequent display to the Surgeon S through the user control unit 1100. The number of instruments 1400 used at one time will generally depend on the diagnostic or surgical procedure and the space constraints within the operating room, among other factors. If it is necessary to change one or more of the instruments 1400 being used during a procedure, an assistant removes the instrument 1400 from the manipulator unit 1200 and replaces it with another instrument 1400 from a tray 1020 in the operating room. Although shown as being used with the instruments 1400, any of the instruments described herein can be used with the MIRS 1000.

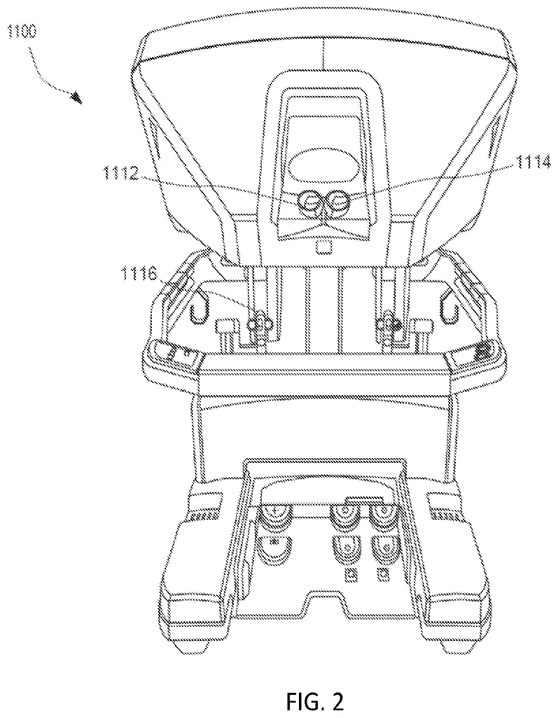

FIG. 2 is a perspective view of the control unit 1100. The user control unit 1100 includes a left eye display 1112 and a right eye display 1114 for presenting the surgeon S with a coordinated stereoscopic view of the surgical site that enables depth perception. The user control unit 1100 further includes one or more input control devices 1116, which in turn cause the manipulator unit 1200 (shown in FIG. 1) to manipulate one or more tools. The input control devices 1116 provide at least the same degrees of freedom as instruments 1400 with which they are associated to provide the surgeon S with telepresence, or the perception that the input control devices 1116 are integral with (or are directly connected to) the instruments 1400. In this manner, the user control unit 1100 provides the surgeon S with a strong sense of directly controlling the instruments 1400. To this end, position, force, strain, or tactile feedback sensors (not shown) or any combination of such sensations, from the instruments 1400 back to the surgeon's hand or hands through the one or more input control devices 1116.

The user control unit 1100 is shown in FIG. 1 as being in the same room as the patient so that the surgeon S can directly monitor the procedure, be physically present if necessary, and speak to an assistant directly rather than over the telephone or other communication medium. In other embodiments, however, the user control unit 1100 and the surgeon S can be in a different room, a completely different building, or other location remote from the patient, allowing for remote surgical procedures.

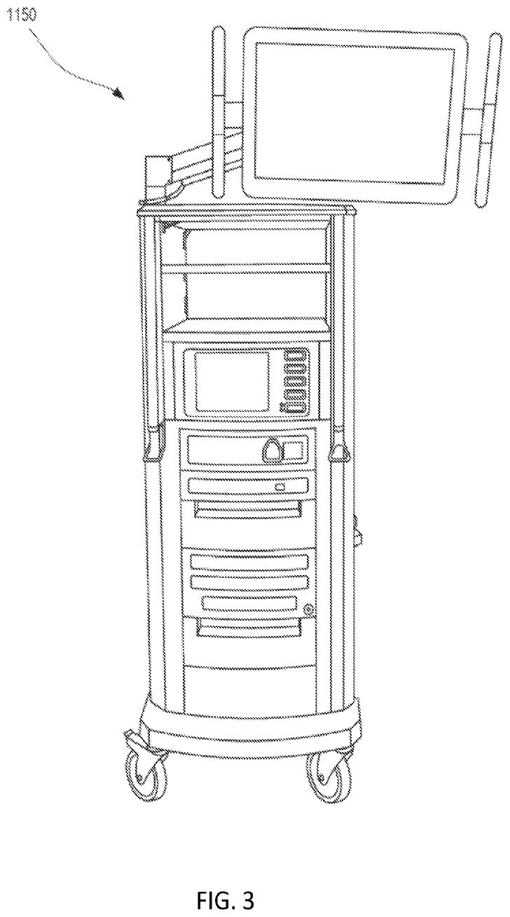

FIG. 3 is a perspective view of the auxiliary equipment unit 1150. The auxiliary equipment unit 1150 can be coupled with the endoscope (not shown) and can include one or more processors to process captured images for subsequent display, such as via the user control unit 1100, or on another suitable display located locally (e.g., on the unit 1150 itself as shown, on a wall-mounted display) and/or remotely. For example, where a stereoscopic endoscope is used, the auxiliary equipment unit 1150 can process the captured images to present the surgeon S with coordinated stereo images of the surgical site via the left eye display 1112 and the right eye display 1114. Such coordination can include alignment between the opposing images and can include adjusting the stereo working distance of the stereoscopic endoscope. As another example, image processing can include the use of previously determined camera calibration parameters to compensate for imaging errors of the image capture device, such as optical aberrations.

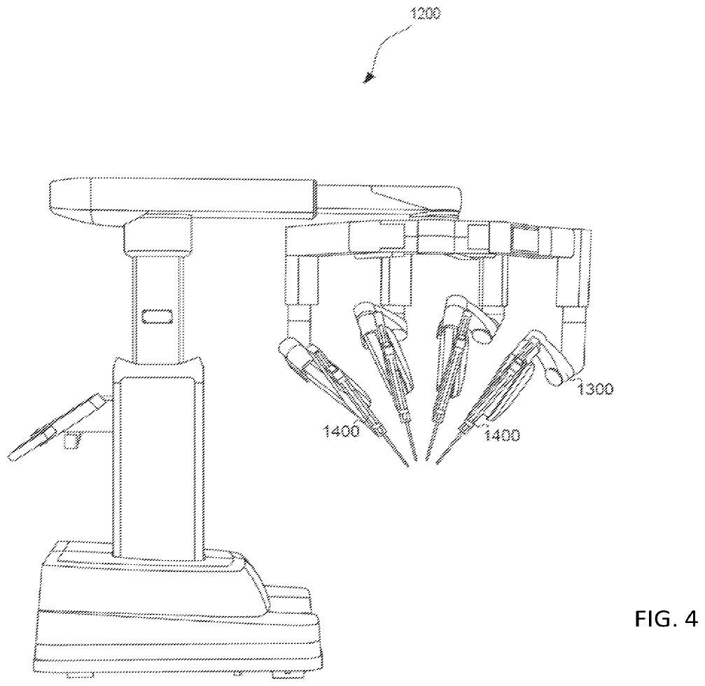

FIG. 4 shows a front perspective view of the manipulator unit 1200. The manipulator unit 1200 includes the components (e.g., arms, linkages, motors, sensors, and the like) to provide for the manipulation of the instruments 1400 and an imaging device (not shown), such as a stereoscopic endoscope, used for the capture of images of the site of the procedure. Specifically, the instruments 1400 and the imaging device can be manipulated by teleoperated mechanisms having one or more mechanical joints. Moreover, the instruments 1400 and the imaging device are positioned and manipulated through incisions or natural orifices in the patient P in a manner such that a center of motion remote from the manipulator and typically located at a position along the instrument shaft is maintained at the incision or orifice by either kinematic mechanical or software constraints. In this manner, the incision size can be minimized.

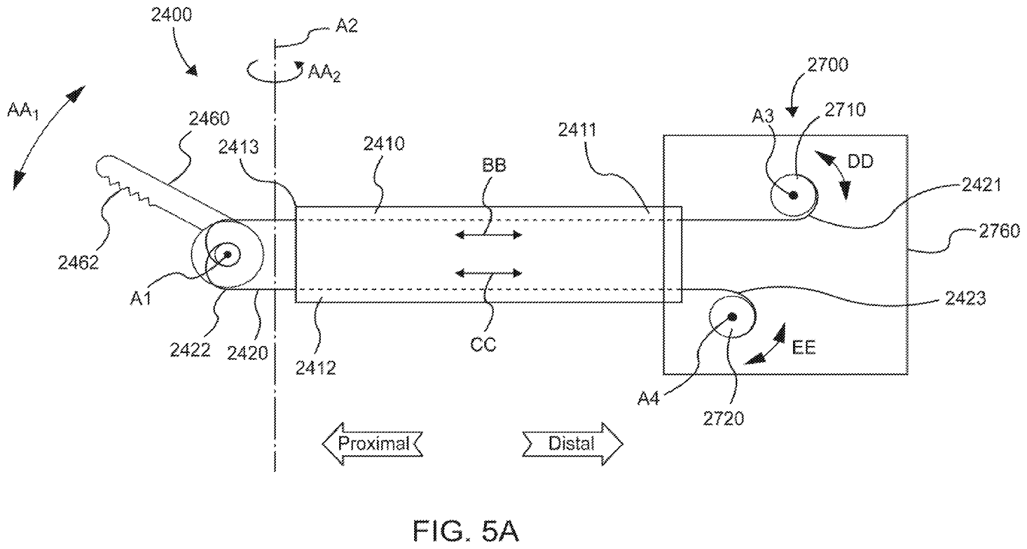

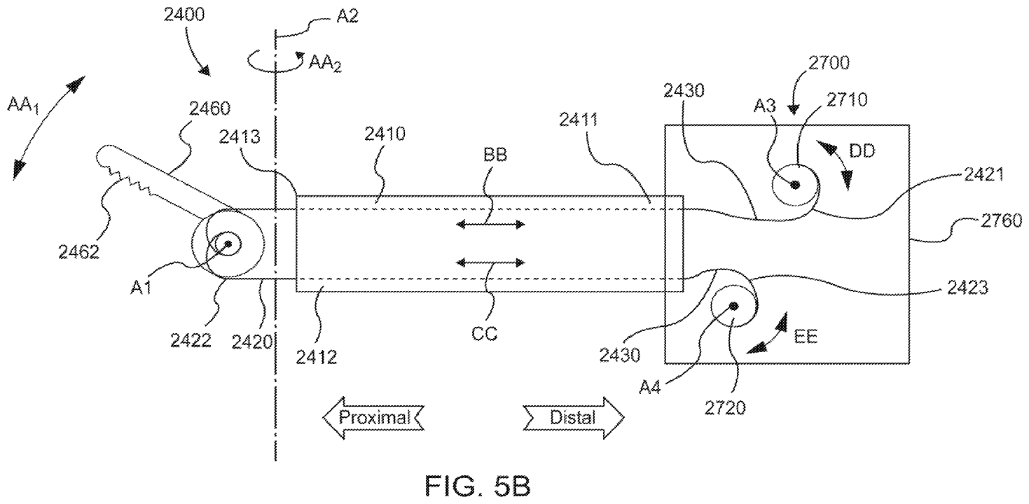

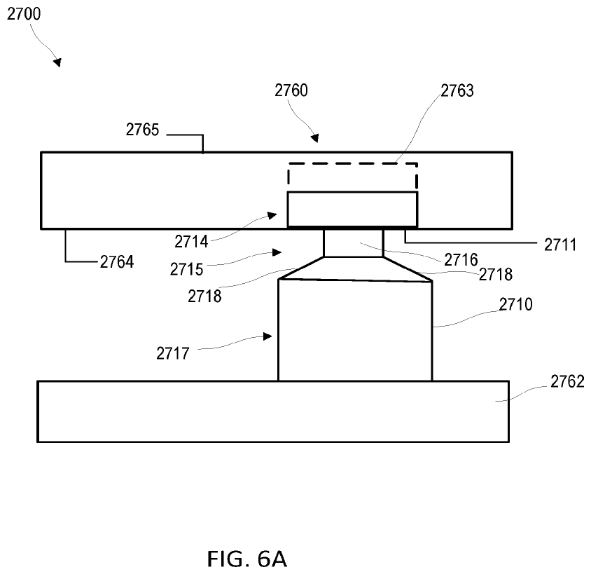

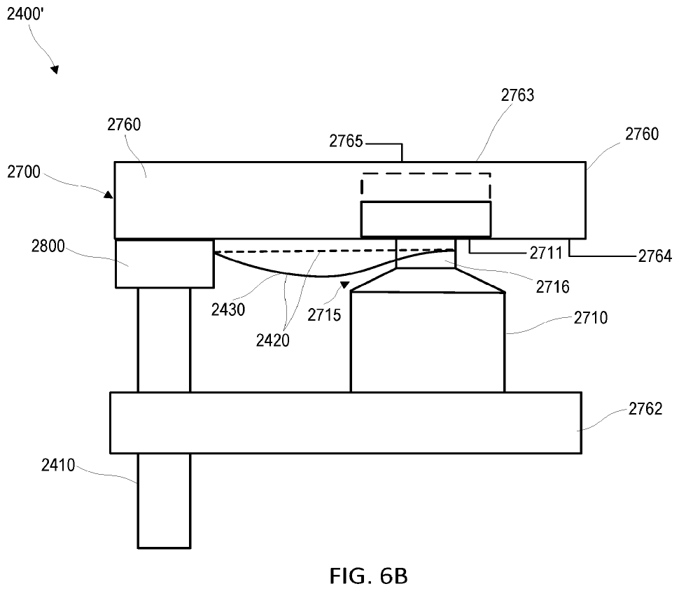

FIGS. 5A-6B are schematic illustrations of a portion of a medical device 2400 according to an embodiment. The instrument 2400 includes a shaft 2410, a cable 2420 (which acts as a first tension member), an end effector 2460, and a mechanical structure 2700. In many embodiments, mechanical structure 2700 functions to receive one or more motor input forces or torques and mechanically transmit the received forces or torques to move an associated one or more components in instrument 2400. For example, one or more electric motors in manipulator unit 1200 provide an input to mechanical structure 2700, which in turn transmits the input to control an instrument 2400 component. In some optional embodiments, mechanical structure 2700 includes one or more motors used to control an instrument 2400 component. In some embodiments, the mechanical structure 2700 (and any of the mechanical structures described herein) can include one or more drive motors to produce the force or torque to move the components of the medical device 2400. In other embodiments, mechanical structure 2700 (and any of the mechanical structures described herein) is devoid of any motors and transmits force or torque from outside the medical device to one or more of the medical device's components. For example, in some embodiments, the mechanical structure 2700 (and any of the mechanical structures described herein) is coupled to a manipulator unit that includes one or more motors that drive an instrument component. The cable 2420 includes a first proximal portion 2421, a second proximal portion 2423 and a distal portion 2422. The first proximal portion 2421 and the second proximal portion 2423 are each coupled to the mechanical structure 2700, and the distal portion 2422 is coupled to the end effector 2460. The shaft 2410 includes a proximal end portion 2411 and a distal end portion 2412 and defines a passageway 2413 that extends lengthwise through the shaft between the proximal and distal end portions.

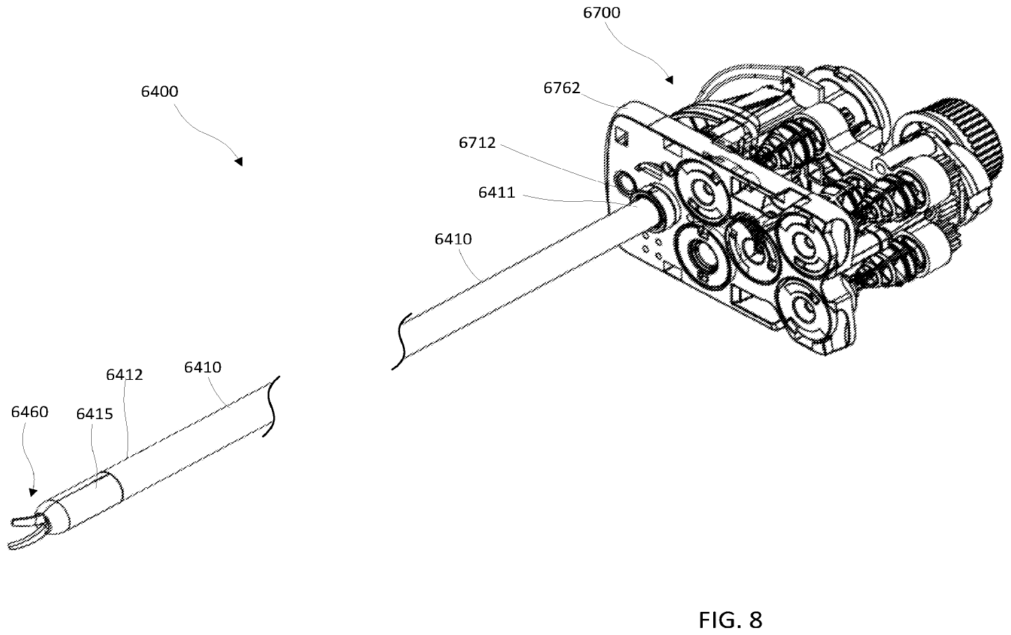

The end effector 2460 is rotatably coupled to the distal end portion 2412 of the shaft 2410 and includes at least one tool member 2462. The instrument 2400 is configured such that movement of the first proximal portion 2421 and the second proximal portion 2423 of the cable 2420 produces movement of the tool member 2462 about a first axis of rotation A1 (which functions as the yaw axis; the term yaw is arbitrary), in a direction of arrows AA₁. In some embodiments, the medical device 2400 can include a wrist assembly including one or more links (not shown in FIGS. 5A-6B) that couples the end effector 2460 to the distal end portion 2412 of the shaft 2410. In such an embodiment, movement of the first proximal portion 2421 and the second proximal portion 2423 of the cable 2420 can also produce movement of the wrist assembly about a second axis of rotation (not shown in FIGS. 5A-6B, but which functions as the pitch axis; the term pitch is arbitrary) or both movement of the wrist assembly and the end effector 2460. An embodiment with a wrist assembly is described herein with reference to FIGS. 8-17B.

The tool member 2462 includes a contact portion 2464, a drive pulley 2470, and a coupling spool 2467. The contact portion 2464 is configured to engage or manipulate a target tissue during a surgical procedure. For example, in some embodiments, the contact portion 2464 can include an engagement surface that functions as a gripper, cutter, tissue manipulator, or the like. In other embodiments, the contact portion 2464 can be an energized portion of the tool member that is used for cauterization or electrosurgical procedures. The end effector 2460 is operatively coupled to the mechanical structure 2700 such that the tool member 2462 rotates relative to shaft 2410 about the first axis of rotation A1 in the direction of the arrow AA₁. In this manner, the contact portion 2464 of the tool member 2462 can be actuated to engage or manipulate a target tissue during a surgical procedure. The tool member 2462 (or any of the tool members described herein) can be any suitable medical tool member. Moreover, although only one tool member 2462 is shown, in other embodiments, the instrument 2400 can include two or more moving tool members that cooperatively perform gripping or shearing functions.

The mechanical structure 2700 includes a chassis 2760, a first capstan 2710, and a second capstan 2720. The chassis 2760 provides the structural support for mounting or supporting and aligning the components of the mechanical structure 2700. For example, openings, protrusions, mounting brackets and the like can be defined in or on chassis 2760. In some embodiments, the chassis 2760 can include multiple portions, such as an upper chassis and a lower chassis. In some embodiments, a housing can optionally enclose at least a portion of the chassis 2760. The first capstan 2710 is mounted to the mechanical structure 2700 (e.g., within the housing 2760) via a first capstan support member (not shown). For example, the first capstan support member can be a mount, shaft, or any other suitable support structure to secure the first capstan 2710 to the mechanical structure 2700.

The second capstan 2720 is mounted to the mechanical structure 2700 (e.g., within the housing 2760) via a second capstan support member (not shown). For example, the second capstan support member can be a mount, shaft, or any other suitable support structure to secure the second capstan 2720 to the mechanical structure 2700. The first capstan 2710 can be operable to be rotated about an axis A3 in a direction DD, as shown in FIGS. 5A and 5B. Th second capstan 2720 can be operable to be rotated about an axis A4 parallel to the axis A3.

The cable 2420 is routed from the mechanical structure 2700 to the end effector 2460 and then back to mechanical structure 2700, and each individual end of the cable is coupled to either the first capstan 2710 or the second capstan 2720 of the mechanical structure 2700. More specifically, the first proximal portion 2421 of the cable 2420 is coupled to the first capstan 2710 of the mechanical structure 2700, the cable 2420 extends from the first capstan 2710 along the shaft 2410, and the distal portion 2422 of the cable 2410 is coupled to the end effector 2460, as described in more detail herein. Although the cable 2420 is shown extending within an interior passageway of the shaft 2410 in FIGS. 5A and 5B, in other embodiments, the cable 2420 can be routed exterior to the shaft 2410. The cable 2420 extends from the end effector 2460 along the shaft 2410 and the second proximal portion 2423 is coupled to the second capstan 2720 of the mechanical structure 2700. In other words, the two ends of a single cable (e.g., 2420) are coupled to and actuated by two separate capstans of the mechanical structure 2700.

More specifically, the two ends of the cable 2420 that are associated with opposing directions of a single degree of freedom are connected to two independent drive capstans 2710 and 2720. This arrangement, which is generally referred to as an antagonist drive system, allows for independent control of the movement of (e.g., pulling in or paying out) each of the ends of the cable 2420. The mechanical structure 2700 produces movement of the cable 2420, which operates to produce the desired articulation movements (pitch, yaw, or grip) at the end effector 2460. Accordingly, as described herein, the mechanical structure 2700 includes components and controls to move a first portion of the cable 2420 via the first capstan 2710 in a first direction (e.g., a proximal direction) and to move a second portion of the cable 2420 via the second capstan 2720 in a second opposite direction (e.g., a distal direction). The mechanical structure 2700 can also move both the first portion of the cable 2420 and the second portion of the cable 2420 in the same direction. In this manner, the mechanical structure 2700 can maintain the desired tension within the cables to produce the desired movements at the end effector 2460.

In other embodiments, however, any of the medical devices described herein can have the two ends of the cable wrapped about a single capstan. This alternative arrangement, which is generally referred to as a self-antagonist drive system, operates the two ends of the cable using a single drive motor.

In addition, in some alternative embodiments, the cable 2420 includes two cable segments, with each cable segment having a distal end portion that is coupled to the end effector 2460 and a proximal end portion wrapped about a capstan—either separate capstans as in the antagonist drive arrangement or a single common capstan in the self-antagonist drive arrangement. Descriptions herein referring to the use of a single cable 2420 incorporate the similar use of two separate cable segments.

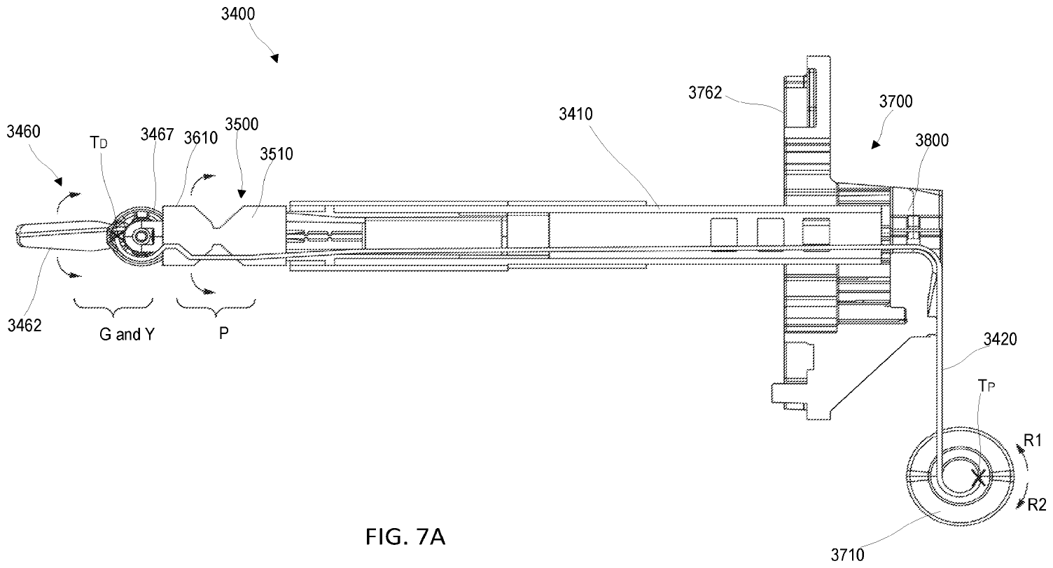

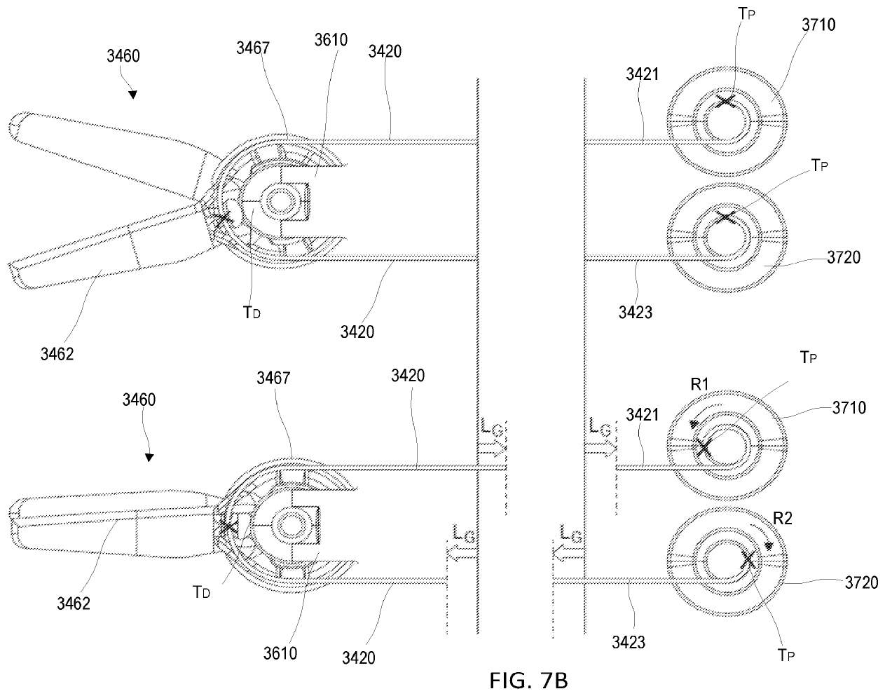

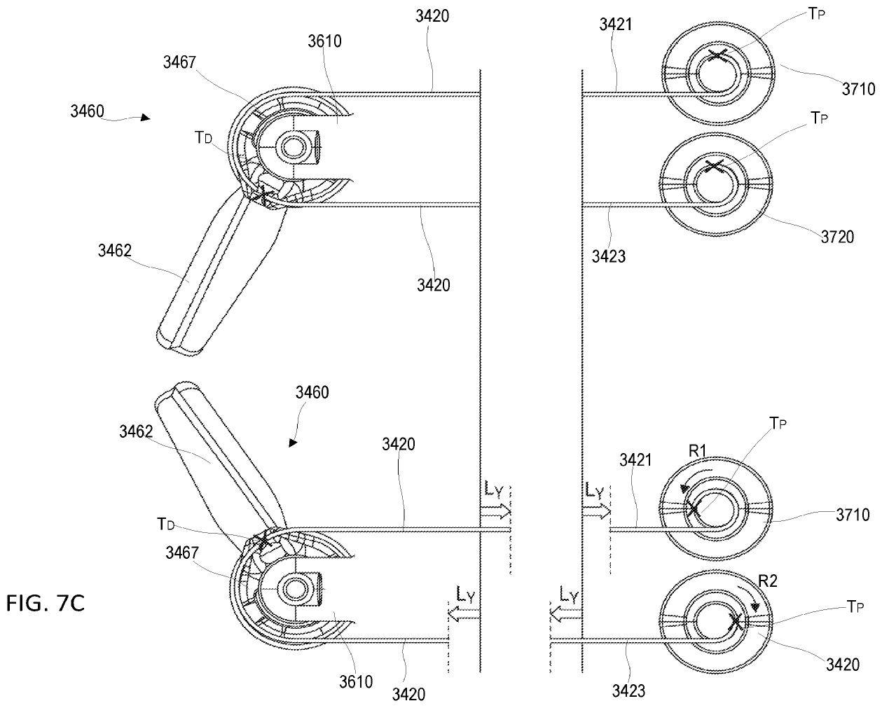

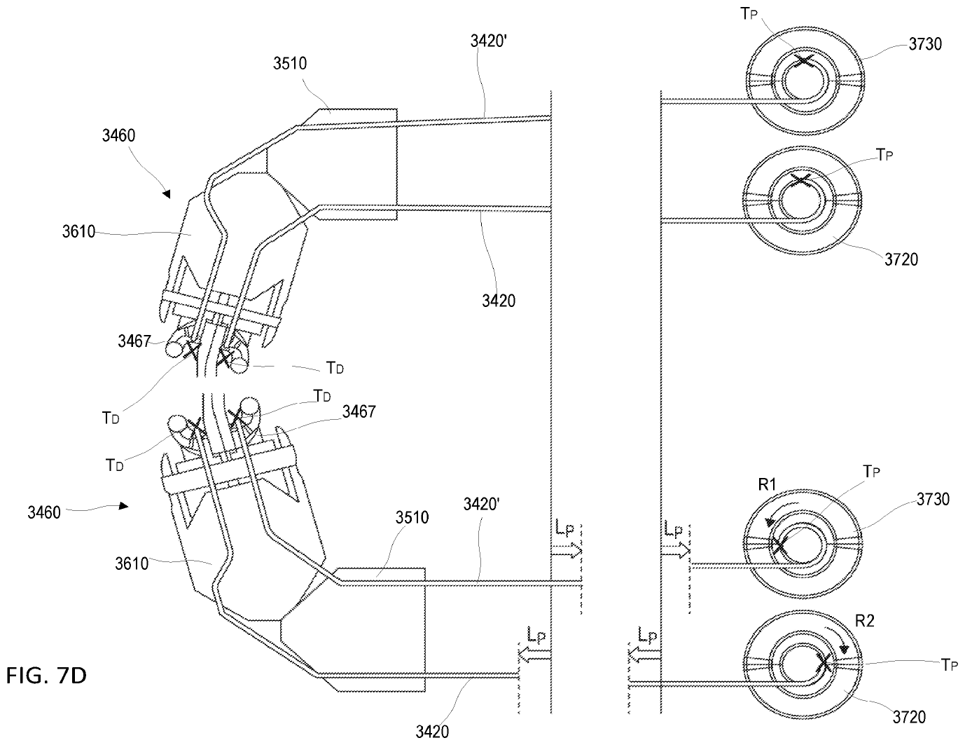

As described above, when the cable(s) 2420 of the medical device 2400 are untensioned, for example when the medical device 2400 is in storage, a slack loop portion 2430 (see e.g., FIG. 5B) of the cable 2420 is formed within the mechanical structure 2700. More details regarding the formation of slack in the cable when untensioned in an antagonistic drive system are described below with reference to FIGS. 7A-7D and are applicable here. The slack loop portion(s) 2430 of the cable 2420 can be, for example, between each of the capstans 2710 and 2720 and a location within the mechanical structure 2700 proximal of the shaft 2410. For example, in some embodiments, a cable guide (2800 shown in the embodiment of FIG. 6B) is located within the mechanical structure 2700, and the cable 2420 is routed through the cable guide and into the shaft 2410. In such an embodiment, the slack loop portion 2430 of the cable 2420 is between the capstans 2710 and 2720 and the cable guide. FIG. 5A illustrates the cable 2420 in a tensioned state (e.g., operational state), and FIG. 5B illustrates the cable 2420 in an untensioned state (e.g., non-operational state) with a slack loop portion 2430 between the capstans 2710 and 2720 and the shaft 2410.

Rather than using mechanisms or other mechanical structures to eliminate or reduce the slack in the cable (e.g., a movable tensioner pulley, a spring-loaded guide surface that moves to maintain cable tension, or a ratchet mechanism to maintain cable tension), the mechanical structure 2700 includes structural features that guide and control the slack loop portion 2430 to be maintained within a desired operational path within the mechanical structure 2700. More specifically, as shown in FIG. 6A, the mechanical structure 2700 includes the first capstan 2710, an upper chassis 2760, and a lower chassis 2762. The capstan 2710 includes an upper portion 2714, a lower portion 2717, and a spool 2715 between the upper portion 2714 and the lower portion 2717. In some embodiments, the upper portion 2714 optionally functions as an anchor portion to secure the cable 2420 to the capstan 2710. The spool 2715 includes a cable wrap surface 2716 (which functions as a drive surface for paying in and out the cable) and a side wall 2718. The cable 2420 is coupled to the first capstan 2710 such that a portion of the first proximal portion 2421 wraps about the cable wrap surface 2716 of the first capstan 2710.

The lower portion 2717 of the first capstan 2710 is supported by the lower chassis 2762, and the upper portion 2714 of the first capstan 2710 is supported within an opening 2763 defined in a bottom 2764 of the upper chassis 2760. In some embodiment, the bottom 2764 of the upper chassis 2760 has a continuous planar surface in which the openings 2763 are defined. In some embodiments, the bottom 2764 of the upper chassis has separated portions with surfaces in which the openings 2763 are defined. For example, bottom 2764 may be defined by surfaces of a support web of braces in upper chassis 2760. In some embodiments, a bottom 2711 of the upper portion 2714 of the first capstan 2710 is within the opening 2763 such that the bottom 2711 is between the bottom 2764 of the upper chassis 2760 and a top surface 2765 of the upper chassis 2760. In other words, the entire upper portion 2714 of the first capstan 2710 is within the opening 2763. In some embodiments, the bottom 2711 of the upper portion 2714 of the first capstan 2710 is positioned to be aligned in the same plane with the bottom 2764 of the upper chassis 2760, and in such embodiments the bottom 2711 of the upper portion 2714 effectively acts as an extension of the upper chassis' bottom 2764. Said another way, in some embodiments, the bottom 2711 of the upper portion 2714 of the first capstan 2710 is positioned even with the bottom 2764 of the upper chassis 2760. Therefore it can be seen that although bottom 2711 may in some embodiments function as an upper side wall of spool 2715, there is no outer perimeter of upper side wall of spool 2715 in a position below the bottom 2764 of the upper chassis, and so the function of such an upper side wall to guide cable slack onto wrap surface 2716 during cable take-up is instead performed by bottom 2764 of upper chassis 2760. As shown in FIGS. 6A and 6B, the side wall 2718 of the spool 2715 slopes away from the bottom 2764 of the upper chassis 2760—i.e., away from the upper chassis 2760 and toward the lower chassis 2762. In some embodiments, the side wall 2718 has a radiused edge to provide a smooth surface for contact with the cable 2420 as described in more detail below.

The medical device 2400 can optionally further include a cable guide 2800 as shown in an alternative embodiment of a medical device 2400′ shown in FIG. 6B. The medical device 2400′ can have the same as or similar components, and it can function the same as, or similar to, the medical device 2400. For example, the medical device 2400′ includes the capstan 2710, upper chassis 2760, and lower chassis 2762 the same as in medical device 2400. In this embodiment, the medical device 2400′ further includes the cable guide 2800 that is coupled within the mechanical structure 2700 and used to guide and redirect the cable 2420 from the capstan 2710 into the shaft 2410. The cable guide 2800 can include any suitable features to guide, redirect, and route the cable into the shaft, either generally straight into the shaft or at an angle from the capstan. For example, another embodiment of a cable guide 6800 is described in more detail below with reference to medical device 6400, and the cable guide 2800 can include similar features. As shown in FIG. 6B, the cable 2420 wrapped on the cable wrap surface 2716 of the spool 2715 of the capstan 2710 extends to the cable guide 2800, where it is redirected through a passageway of the shaft 2410 and is then operatively connected to the end effector 2460. In some embodiments, the cable 2420 is wrapped on the spool 2715 such that the cable 2420 is routed about the cable wrap surface 2416 of the spool 2715 by no more than two revolutions.

When the cable 2420 is in an untensioned state (i.e., the instrument and cable are in a pre-operative state, such as during storage), a slack loop portion 2430 exists in the cable 2420 between the cable guide 2800 and the spool 2715. When the capstan 2710 rotates, the cable 2410 is wound about the cable wrap surface 2416 of the spool 2145, and the cable 2420 transitions from the untensioned state to a tensioned state as shown by the cable 2420 shown in dashed line in FIG. 6B. During this transition, the slack loop 2430 of the cable 2420 is guided and maintained between the bottom 2764 of the upper chassis 2760 and the side wall 2718 of the spool 2715 to ensure that the slack loop portion 2430 of the cable 2420 is properly wound around the spool and also routed to the cable guide 2800. In other words, the bottom 2764 and the side wall 2718 limit the likelihood that the slack loop portion 2430 inadvertently becomes wrapped, pinched, or trapped in an undesirable portion of the mechanical structure 2700. In some embodiments, this arrangement can accommodate large amounts of cable slack in the untensioned state. For example, in some embodiments, the slack loop portion 2430 can be sized such that the portion of the cable 2420 within the mechanical structure 2700 is between 2 percent and 15 percent of the total length of the cable 2420. In some embodiments, the slack loop portion 2430 can be sized such that the portion of the cable 2420 within the mechanical structure 2700 is between 3 percent and 10 percent of the total length of the cable 2420.

In some embodiments, the medical device further includes a first bearing (not shown in FIGS. 5A-6B; see additional description below) that supports the upper portion 2714 of the capstan 2710 within the upper chassis 2760 and surrounds the portion of the cable 2420 coupled to the upper portion 2714 of the capstan 2710. Thus, the first bearing can assist in maintaining the cable 2420 coupled to the capstan 2710. In some embodiments, the medical device can further include a second bearing (not shown in FIGS. 5A-6B; see additional description below) that can support the lower portion 2717 of the capstan 2710 within the lower chassis 2762. In some embodiments, the first bearing can be, for example, a rolling-element bearing (e.g., a ball, needle, etc.), and the second bearing can be, for example, a journal bearing. In some embodiments, the spool 2715 of the capstan 2710 is located adjacent the upper chassis 2760 such that when tension in the cable 2420 causes a lateral load on the capstan 2710, a portion of the lateral load on the first bearing is larger than a portion of the lateral load on the second bearing. Embodiments of a first bearing and a second bearing are described below in more detail with reference to medical device 6400.

With the cable 2420 coupled to the mechanical structure 2700 and to the end effector 2460, rotational movement produced by the first capstan 2710 causes the first proximal portion 2421 of the cable 2420 to move in a direction BB (e.g., proximally or distally depending on the direction of rotation), as shown in FIGS. 5A and 5B. Similarly, rotational movement produced by the second capstan 2720 causes the second proximal portion 2423 of the cable 2420 to move in the direction CC (e.g., proximally or distally depending on the direction of rotation), as shown in FIGS. 5A and 5B. Said another way, the first capstan 2710 can be operable to produce rotational movement about the axis A3, and the second capstan 2720 can similarly be operable to produce rotational movement about an axis A4 parallel to the axis A3. Thus, the first capstan 2710 can rotate in the direction of arrows DD and the second capstan 2720 can rotate in the direction of arrows EE in FIGS. 5A and 5B. For example, when the first capstan 2710 rotates about the axis A3 in a first direction (clockwise or counter-clockwise), the second capstan 2720 rotates about the axis A4 in either the same or the opposite direction (clockwise or counter-clockwise). Thus, as one of the capstans 2710, 2720 pays out the cable 2420, the other of the capstans 2710, 2720 pays in the cable 2420. Depending on how the cables are routed to the various capstans, it doesn't matter what direction each of the individual capstans rotates as long as the desired individual cable pay-in or pay-out is performed to perform the desired end effector motion—grip, yaw; or pitch—either alone or in combination.

With each of the ends of the cable 2420 coupled to a separate capstan, the movement of a first portion of the cable 2420 can be directly controlled by one capstan (e.g., first capstan 2710) and movement of a second portion of the cable 2420 can be directly controlled by the other capstan (e.g., second capstan 2720)). Thus, the control of motion of the end effector 2460 in one direction is controlled by one capstan, and the control of motion of the end effector 2460 in the other direction is controlled by the other capstan. In this antagonist system, however, when the first capstan 2710 is controlling motion (i.e., applying tension to pull in the first proximal portion 2421 of the cable 2420), the second proximal portion 2423 of the cable is also under tension applied by the second capstan 2720. Maintaining tension applied by the non-driving capstan (i.e., the second capstan 2720) allows the non-driving capstan to immediately function as the driving capstan with no hysteresis in end effector control. The differing levels of tension applied by each capstan can also lead to improved control of the overall movement of the cable. Thus, better control of the overall movement of the end effector 2460 can be achieved. For example, accurate rotation in yaw around axis A1 can be controlled. The first capstan 2710 can be actuated to produce a rotational movement about the axis A3 in the direction of the arrow DD such that the first proximal portion 2421 of the cable is moved in a first direction along arrows BB. Simultaneously, the second capstan 2720 can be actuated to produce rotational movement about an axis parallel to the axis A3 in an opposite direction as the first capstan 2710 such that the second proximal end portion 2723 of the cable 2420 is moved in an opposite direction as the first proximal portion 2423 along arrows CC. Thus, the opposite movement of the first proximal portion 2421 and the second proximal portion 2423 causes the end effector 2460 to rotate (via the cable 2420 connection to the end effector 2460) about the rotational axis A1 (e.g., yaw movement).

In a similar way, accurate rotation in pitch around a second axis A2 (e.g., pitch; orthogonal to the yaw axis A1 described above) can be controlled. As described above, the first capstan 2710 can be actuated to produce a rotational movement about the axis A3 in the direction of the arrow DD, while simultaneously the second capstan 2720 can be actuated to produce rotational movement about the axis A4 parallel to the axis A3 in the direction of the arrow EE such that the first proximal portion 2421 of the cable and the second proximal portion 2423 of the cable 2420 are moved together in the same direction (along arrows BB and CC, respectively). The movement of the first proximal portion 2421 and the second proximal portion 2423 in the same direction causes the end effector 2460 (or a wrist mechanism) to rotate (via the cable 2420 connection to the end effector 2460) about a second rotation axis A2 in the direction of arrow AA₂ (e.g., pitch movement). Persons of skill in the art will understand that this action controls rotation around the second axis A2 in a first direction, and a similar action by an additional cable (or cable segments) (not shown) controls rotation around the second axis A2 in a second direction opposite the first direction. Thus an antagonistic control relationship between the cable portions 2420 acting together and the additional cable is used to accurately control end effector rotation in pitch. Alternatively, a resiliency such as a spring may be used to act against cable portions 2420 to urge rotation around the second axis A2 in a direction opposite to the direction urged by cable portions 2420. Thus, the combination of the first capstan 2710, the second capstan 2720, and the single cable 2420 are operable to control the end effector 2460 of instrument 2400 in at least 2 DOFs (e.g., pitch and yaw).

The cable 2420, and any of the cables described herein can be made from any suitable materials. For example, in some embodiments, any of the cables described herein can be formed from an ultra-high molecular weight polyethylene (UHMWPE) fiber. In some embodiments, any of the cables described herein can be constructed from a single monofilament. In other embodiments, any of the cables described herein can be constructed from multiple cofilament strands, laid or woven (or both), or thermally fused, or otherwise combined to form the cable. In some embodiments, the cable 2420 or any of the cables described herein can include an optional outer sheath, coating, or other surface treatment to increase the frictional characteristics of the cable. Such increased frictional characteristics help facilitate having the cable 2420 wrapped to the capstan without slipping and without the need for an additional retention feature.

In some embodiments, the cable 2420 and any of the cables described herein can be made from a material having suitable temperature characteristics for use with cauterizing instruments. For example, such materials include liquid crystal polymer (LCP), aramid, para-aramid, and polybenzobisoxazole fiber (PBO). Such materials can provide frictional characteristics that increase the ability for friction coupling and improve holding ability, for example for coupling the cable 2420 to the capstan 2710 and end effector 2460. Such ability can also improve slip characteristics (e.g., help prevent the cable from slipping) during operation of the medical device. Such materials may or may not need a coating or other surface treatment to increase the frictional characteristic.

FIGS. 7A-7D are structural illustrations of another embodiment of a medical device illustrating the actuation and function modes of an antagonist drive system. The medical device 3400 can be configured the same or similar to, and functions the same as or similar to, the medical devices described herein. The medical device 3400 includes a proximal mechanical structure 3700, a shaft 3410 having a proximal end portion coupled to the mechanical structure 3700 and a distal end portion coupled to a wrist assembly 3500, and an end effector 3460 coupled to the wrist assembly 3500. The mechanical structure includes a lower chassis 3762. a first capstan 3710, a second capstan 3720 (see FIGS. 7B-7D), and a cable guide 3800. The wrist assembly 3500 includes a proximal first link 3510 and a distal second link 3610. The end effector 3460 includes a tool member 3462 and a coupling spool 3467. An illustrative cable 3420 extends between the capstans and the end effector 3460 and includes a first proximal portion 3421 coupled to the first capstan 3710, a second proximal portion 3423 coupled to the second capstan 3720 and a distal portion coupled to the coupling spool 3467 of the tool member 3462. In practice, two cables and four capstans are used, with each cable coupled between two capstans as illustrated. Specifically, one cable (e.g., cable 3420) is coupled to a first tool member 3462 of the end effector 3460, and the second cable (see, e.g., cable 3420′ in FIG. 7D) is coupled to a second tool member of the end effector 3460.

More specifically, the first proximal portion 3421 of the cable 3420 is coupled to the first capstan 3710, is routed through the cable guide 3800, into an interior passageway of the shaft 3410, and to the end effector 3460, where the distal portion of the cable is coupled to the coupling spool 3467. The second proximal portion 3423 of the cable 3420 extends proximally back through the shaft 3410, though the cable guide 3800, and to the second capstan 3720. Although the cable 3420 is described as extending within an interior passageway of the shaft 3410, in other embodiments the cable 3420 can be routed exterior to the shaft 3410. Thus, the two ends of a single cable (e.g., 3420) are coupled to and actuated by two separate capstans (3710, 3720) of the mechanical structure 3700.

More specifically, the two opposite ends of the cable 3420 are associated with opposing directions of a single degree of freedom and are connected to two independent drive capstans 3710 and 3720. As described above, this arrangement is generally referred to as an antagonist drive system, and it allows for independent control of the movement of (e.g., pulling in or paying out) each of the ends of the cable 3420. The mechanical structure 3700 produces tension in and movement of the cable 3420, which operates to produce the desired articulation movements, pitch P, yaw Y, or grip G at the end effector 3460 (see arrows P, G, and Y in FIG. 7A). Accordingly, as described herein, the mechanical structure 3700 includes components and controls to move a first proximal portion 3421 of the cable 3420 via the first capstan 3710 in a first direction (e.g., a proximal direction) and to move a second proximal portion 3423 of the cable 3420 via the second capstan 2720 in a second opposite direction (e.g., a distal direction). The mechanical structure 3700 can also move both the first portion of the cable 3420 and the second portion of the cable 3420 in the same direction. In this manner, the mechanical structure 3700 can maintain the desired tension within the cable portions to produce the desired movements at the end effector 3460. As described above, each of the proximal cable portions is maintained under tension, and the cable movement can be controlled by controlling the difference in tension between the two portions. Thus, movement in one direction results when one of the capstans produces a higher tension in its cable portion than the other capstan produces in its cable portion. Cable movement in the opposite direction results when the other capstan produces higher tension in its cable portion.

As described above, when the cable 3420 is in an untensioned state (e.g., when the medical device 3400 is in storage, or before the medical device 3400 is engaged on a manipulator for operation) a slack loop portion (not shown in FIGS. 7A-7D) of the cable 3420 is formed within the mechanical structure 3700. The slack loop portion of the cable 3420 can be, for example, between each of the capstans 3710 and 3720 and a location within the mechanical structure 3700 and proximal of the shaft 3410. For example, in some embodiments the slack loop portion of the cable 3420 is between the capstans 3710 and 3720 and the cable guide 3800. During operation of the medical device 3400, when one of the capstans 3710, 3720 rotates in a first direction (e.g., either the direction of arrow R1 or arrow R2) as shown in FIG. 7A, the cable 3410 is wound about a portion of the capstan 3710, 3720, and the cable 3420 transitions from the untensioned state to a tensioned state.

The cable 3420 has a length L_{T} that is a minimum fixed length L_{F} plus the length associated with the slack loop L_{S}. The minimum fixed length is the distance between a proximal termination point T_{P} and a distal termination point T_{D}, as shown in FIGS. 7A-7D. This fixed length is determined based on physical dimensions of the device, such as the length of the shaft 3410, location of the capstans 3710, 3720, etc. The length associated with the slack loop (when the capstans 3710, 3720 are not driven to rotate) is determined by the linear translation of the cable 3420 corresponding to the movement of the wrist assembly 3500 in grip L_{G}, yaw L_{Y}, and pitch L_{P} as shown in FIGS. 7B-7D. Thus, the total length of the cable is L_{T}=L_{F}+(L_{G}+L_{Y}+L_{P}). In other words, the cable length must be long enough to reach and control all extreme range of motion positions of the end effector 3460 and wrist assembly 3500 combined when the instrument is in an operational state. And so, the maximum cable slack length Ls is equal to L_{G}+L_{Y}+L_{P}, and this maximum slack length must be reliably wound up on one or both of the capstans as the instrument transitions from a pre-operational state, in which the cable is not under tension, to an operational state, in which the cable is under full operational tension.

FIGS. 7B-7D illustrate the cable travel during movement of the end effector 3460 and wrist assembly 3500 in an antagonist drive system. FIG. 7B illustrates the cable travel during grip movement. The top portion of the FIG. 7B illustrates the end effector 3460 in a grip open position. When the capstan 3710 is rotated in the direction R1, and the capstan 3720 is rotated in the direction R2, to close the grip, the cable 3420 is spooled onto the capstan 3710, and cable length is let out of the capstan 3720. Thus, the length L_{G} associated with the grip movement between the proximal termination point T_{P} of the capstan 3710 and the distal termination point T_{D} at the end effector 3460 is the same as the L_{G} associated with the grip movement between the proximal termination point T_{P} of the capstan 3720 and the distal termination point T_{D} at the end effector.

FIG. 7C illustrates the cable travel during yaw movement of the end effector 3460. The top portion of the FIG. 7C illustrates the end effector 3460 in a yaw left position. When the capstan 3710 is rotated in the direction R1, and the capstan 3720 is rotated in the direction R2 for a yaw right movement, the cable 3420 is spooled onto the capstan 3710, and cable length is let out of the capstan 3720. Thus, the length L_{Y} associated with the yaw movement between the proximal termination point T_{P} of the capstan 3710 and the distal termination point T_{D} at the end effector 3460 is the same as the L_{Y} associated with the yaw movement between the proximal termination point T_{P} of the capstan 3720 and the distal termination point T_{D} at the end effector.

FIG. 7D illustrates the cable travel during pitch movement of the wrist assembly 3500. In this embodiment, pitch is controlled by selective movement of (and tension within) the first cable 3420 and a second cable 3420′ (i.e., the cable that is coupled to the second tool member of the end effector 3460). The second cable 3420′ has a first proximal end portion 3421′ coupled to a third capstan 3730 and a second proximal end portion (not shown) coupled to a fourth capstan (not shown). The top portion of FIG. 7D illustrates the wrist assembly 3500 in a pitch down position. When the capstan 3730 is rotated in the direction R1, and the capstan 3720 is rotated in the direction R2 for a pitch up movement, the cable 3420 is spooled onto the capstan 3710, and cable length is let out of the capstan 3720. Thus, the length L_{P} associated with the pitch movement between the proximal termination point T_{P} of the capstan 3710 and the distal termination point T_{D} at the end effector 3460 is the same as the L_{P} associated with the pitch movement between the proximal termination point T_{P} of the capstan 3720 and the distal termination point T_{D} at the end effector.

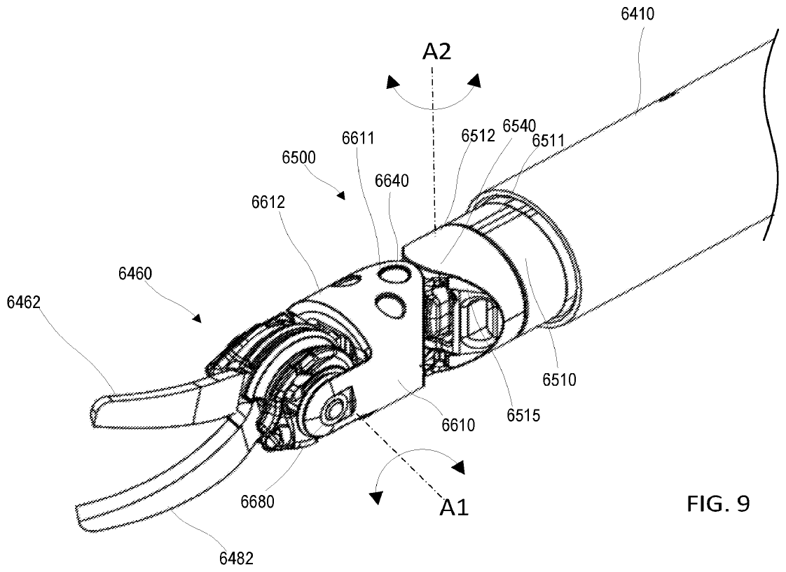

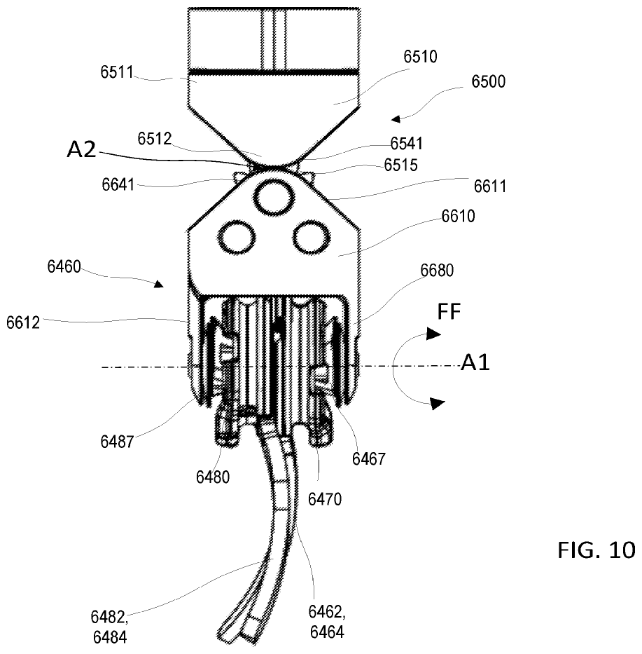

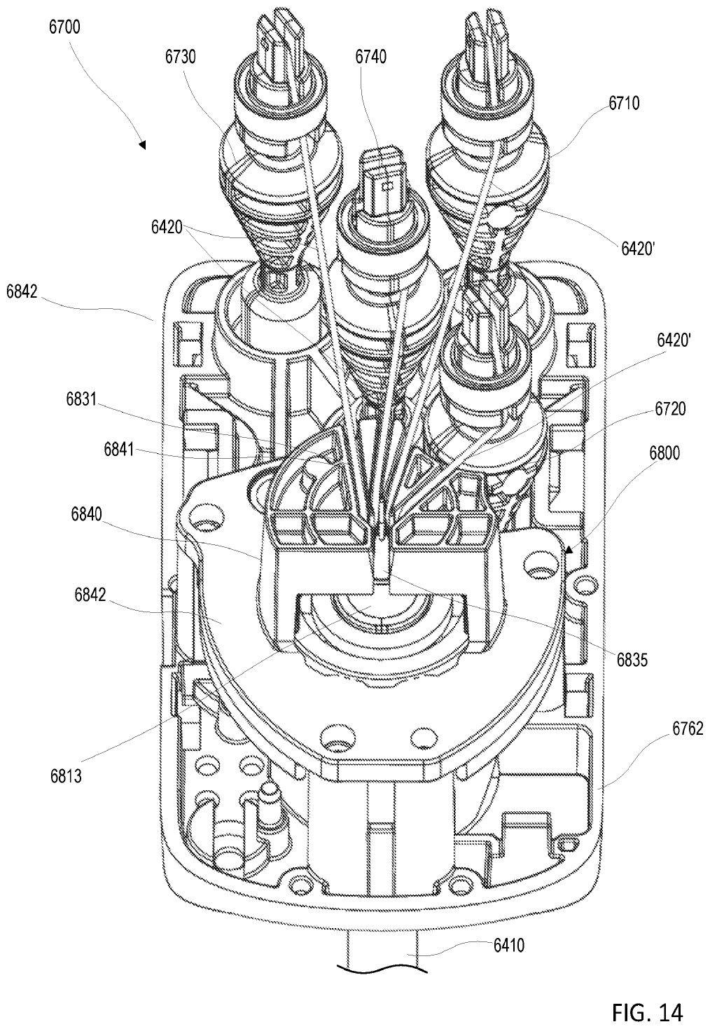

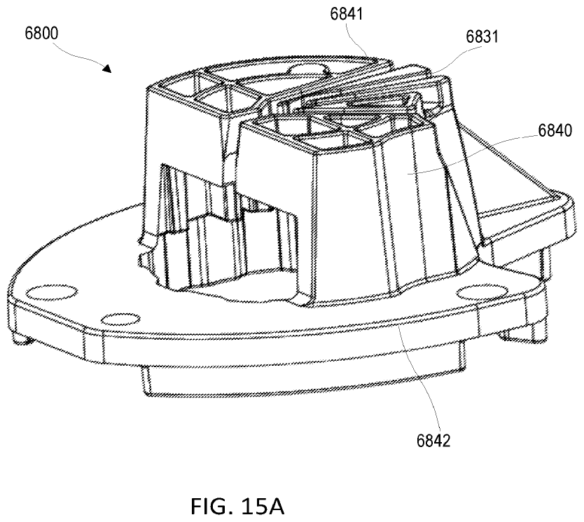

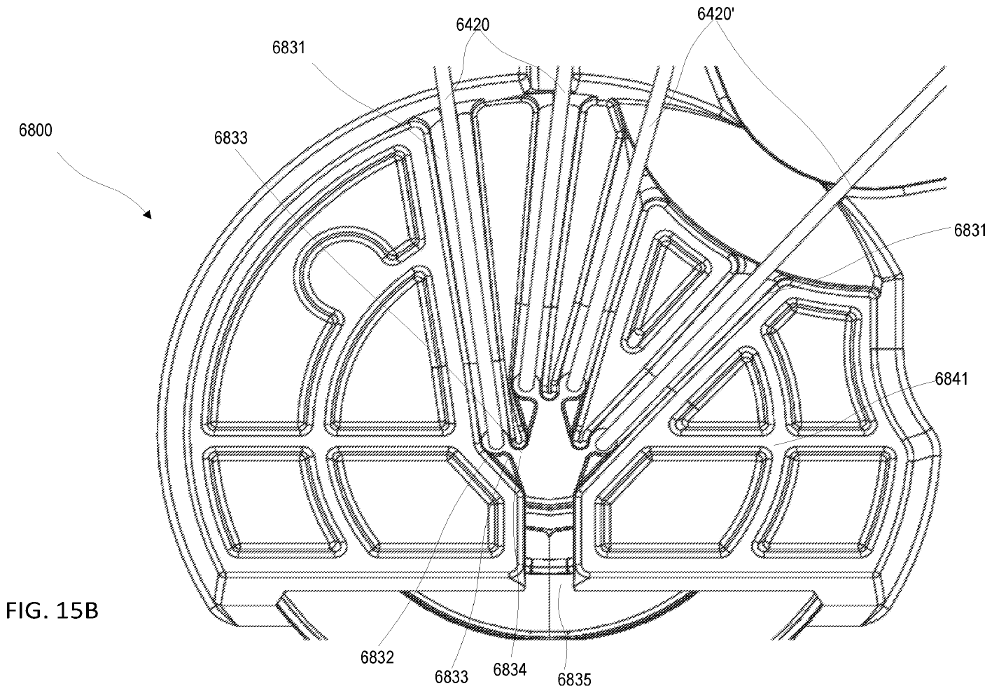

FIGS. 8-17 are various views of an instrument 6400, according to an embodiment. In some embodiments, the instrument 6400 or any of the components therein are optionally parts of a surgical system that performs surgical procedures, and which can include a manipulator unit, a series of kinematic linkages, a series of cannulas, or the like. The instrument 6400 (and any of the instruments described herein) can be used in any suitable surgical system, such as the MIRS system 1000 shown and described above. The instrument 6400 includes a proximal mechanical structure 6700, a shaft 6410, a distal wrist assembly 6500, a distal end effector 6460, and a distal cover 6415. Although not shown, the instrument 6400 also includes a first cable 6420 and a second cable 6420′ (see e.g., FIGS. 14 and 15B) that couple the proximal mechanical structure 6700 to the distal wrist assembly 6500 and end effector 6460. The instrument 6400 is configured such that movement of the first cable 6420 and second cable 6420′ produces rotation of the end effector 6460 about a first axis of rotation A1 (see FIGS. 9 and 10, which functions as a yaw axis, the term yaw is arbitrary), rotation of the wrist assembly 6500 about a second axis of rotation A2 (see FIGS. 9 and 10, which functions as a pitch axis), a cutting rotation of the tool members of the end effector 6460 about the first axis of rotation A1, or any combination of these movements. Changing the pitch or yaw of the instrument 6400 can be performed by manipulating the cables in a similar manner as that described above for the instrument 2400 and 3400. Thus, the specific movement of each of the cables to accomplish the desired motion is not described below.

The shaft 6410 can be any suitable elongated shaft that couples the wrist assembly 6500 to the mechanical structure 6700. Specifically, the shaft 6410 includes a proximal end 6411 that is coupled to the mechanical structure 6700, and a distal end 6412 that is coupled to the wrist assembly 6500 (e.g., a proximal link of the wrist assembly 6500). The shaft 6410 one or more passageways, through which the cables and other components (e.g., charged electrical wires, ground wires, or the like) can be routed from the mechanical structure 6700 to the wrist assembly 6500. In the example shown, the optional cover 6415 (see FIG. 8) is positioned over the wrist assembly 6500 and at least a portion of the end effector 6460.

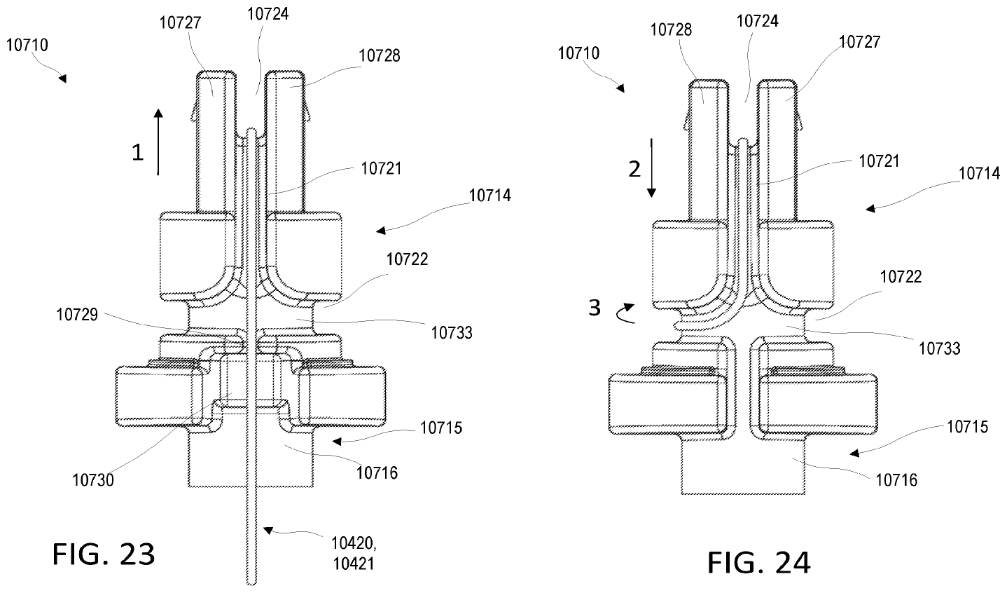

The first cable 6420 and the second cable 6420′ each include a first proximal portion, a second proximal portion, and a distal portion. As described above for cable 2420, the first proximal end portion and the second proximal end portion are each coupled to the mechanical structure 6700 in the same manner as described above for mechanical structure 2700 of instrument 2400 and as described in more detail below with reference to FIGS. 23-29. In some embodiments, the cables 6420 and 6420′ can be constructed from a polymer as described above for the cable 2420.

Referring to FIGS. 9 and 10, the wrist assembly 6500 (also referred to as a joint assembly) includes a first link 6510, a second link 6610 and a third link 6515. The first link 6510 has a proximal portion 6511 and a distal end portion 6512. The proximal end portion is coupled to the shaft 6410. The proximal portion 6511 can be coupled to the shaft 6410 via any suitable mechanism. For example, in some embodiments, the proximal portion 6511 can be matingly disposed within a portion of the shaft 6410 (e.g., via an interference fit). In some embodiments, the proximal portion 6511 can include one or more protrusions, recesses, openings, or connectors that couple the proximal portion 6511 to the shaft 6410. For example, in some embodiments, a pin extends through a hole in the proximal portion 6511 and a corresponding hole in the shaft 6410. In some embodiments, the proximal portion 6511 can be welded, glued, or fused to the shaft 6410.

The distal end portion 6512 includes a joint portion 6540 that is rotatably coupled to a mating joint portion 6640 of the second link 6610 as described in more detail below. The second link 6610 has a proximal portion 6611 and a distal end portion 6612. The proximal portion 6611 includes a joint portion 6640 that is rotatably coupled to the joint portion 6540 of the first link 6510 to form the wrist assembly 6500 having the second axis of rotation A2 about which the second link 6610 rotates relative to the first link as shown in FIGS. 9 and 10. The wrist assembly 6500 can include any suitable coupling mechanisms. In this embodiment, the first link 6510 is coupled to the third link 6515 via a pinned joint and the second link 6610 is coupled to the third link 6515 via a pinned joint. In this manner, the third link 6515 maintains the coupling between the first link 6510 and the second link 6610 during rotation of the second link 6610 relative to the first link 6510.

Further, as described above, the distal end portion 6512 of the first link 6510 includes a joint portion 6540 that is rotatably coupled to a mating joint portion 6640 at the proximal end portion 6611 of the second link 6610. Specifically, the joint portion 6540 includes a series of teeth (not shown) that are spaced apart by recesses (not shown), and the joint portion 6640 includes a series of teeth (not shown) that are spaced apart by recesses (not shown). The series of teeth and recesses can be similar to those shown and described in U.S. Patent Application Pub. No. US 2017/0120457 A1 (filed Feb. 20, 2015), entitled “Mechanical Wrist Joints with Enhanced Range of Motion, and Related Devices and Methods,” or to those shown and described in International Application No. PCT/US18/64721 (filed Dec. 10, 2018), entitled “Medical Tools Having Tension Bands,” each of which is incorporated herein by reference in its entirety. The teeth of the first link 6610 engage the teeth of the second link 6610 during rotation of the second link 6610 relative to the first link 6510. In addition, the joint portion 6540 has a curved surface 6541 that engages a curved surface 6641 of the joint portion 6640 during rotation of the second link 6610 relative to the first link 6510. Because the wrist joint (i.e., the joint between the first link 6510 and the second link 6610) is not a pinned joint, the second axis A2 will move relative to the first link 6510 during rotation of the second link 6610. In other words, the location of the second axis A2 will move (for example, as viewed in a top view) with the rolling movement of the second link 6610 relative to the first link 6510.

As shown in FIGS. 9 and 10, the end effector 6460 is coupled to the second link 6610. More specifically, the distal end portion 6612 of the second link 6610 includes a connector 6680 that is coupled to the end effector 6460 such that the end effector 6460 (e.g., tool members of the end effector) rotates relative to the wrist assembly 6500 about the first axis of rotation A1 (see, e.g., FIG. 10). The second axis of rotation A2 is non-parallel to the first axis of rotation A1. The first axis A1 also functions as a cutting axis as tool members rotate in opposition to each other as described in more detail below. Thus, the instrument 6400 provides at least three degrees of freedom (i.e., yaw motion about the first axis of rotation A₁, pitch rotation about the second axis of rotation A2, and a cutting motion about the first axis of rotation A1). In alternative embodiments, depending on the type of end effector, rather than a cutting motion, the end effector can provide motion for other actions, such as, for example, grasping, cauterizing, etc. about the first axis of rotation A1. The connector 6680 can be any suitable connector to rotatably couple the end effector 6460 to the wrist assembly 6500. For example, in some embodiments, the first link 6510 can include a clevis and a pin, such as the pinned joints shown and described in U.S. Pat. No. 9,204,923 B2 (filed Jul. 16, 2008), entitled “Medical Device Electronically Energized Using Drive Cables,” which is incorporated herein by reference in its entirety.

As shown in FIGS. 9 and 10, the end effector 6460 includes a first tool member 6462 and a second tool member 6482. The first tool member 6462 includes a contact portion (not shown), a drive pulley 6470 and a coupling spool 6467. The contact portion of the first tool member 6462 is configured to engage or manipulate a target tissue during a surgical procedure. For example, in this embodiment, the contact portion of the first tool member 6462 includes an engagement surface that functions as a cutter (e.g., a cutting blade). In other embodiments, the contact portion 6464 can function as a gripper, tissue manipulator, or the like, or can be an energized tool member that is used for cauterization or electrosurgical procedures. The second tool member 6482 includes a contact portion (not shown), a drive pulley 6480 and a coupling spool 6487. The contact portion of the second tool member 6482 is configured to engage or manipulate a target tissue during a surgical procedure. For example, in this embodiment, the contact portion of the second tool member 6482 includes an engagement surface that functions as a cutter (e.g., a cutting blade). In other embodiments, the contact portion can function as a gripper, tissue manipulator, or the like, or it can be an energized tool member that is used for cauterization or other electrosurgical procedures. In this embodiment, the drive pulley 6470 and coupling spool 6467 can be formed as an integral or monolithic component that is welded (or otherwise coupled) to the contact portion of the first tool member 6462, and the drive pulley 6480 and coupling spool 6487 can be formed as an integral or monolithic component that is welded (or otherwise coupled) to the contact portion of the second tool member 6482. In some embodiments, the drive pulley 6470 and coupling spool 6467 and the contact portion of the first tool member 6462 are made as a single monolithic piece, and the drive pulley 6480 and coupling spool 6487 and the contact portion of the second tool member 6482 are made as a single monolithic piece. In some embodiments, the contact portions of the first tool member 6462 and the second tool member 6482 can each be formed as two parts. In some embodiments, the drive pulleys 6470, 6480 and coupling spools 6467, 6487 are made with a metallic material and formed, for example, through a metal injection molding process. In some embodiments, the drive pulley 6470, 6480 and coupling spools 6467, 6487 can be configured the same as or similar to, and function the same as or similar to, the drive pulleys and coupling spools shown and described in International PCT Application No. PCT/US2021/017840 (filed Feb. 12, 2021), which is incorporated herein by reference in its entirety.

The end effector 6460 can be operatively coupled to the mechanical structure 6700 such that the tool members 6462 and 6482 rotate about the first axis of rotation Al. For example, the drive surface 6471 of the drive pulley 6470 is configured to engage the first cable 6420 such that a tension force exerted by the first cable 6420 along the drive surface 6471 produces a rotation torque about the first axis A1. Similarly, the drive surface 6481 of the drive pulley 6480 is configured to engage the second cable such that a tension force exerted by the second cable along the drive surface 6481 produces a rotation torque about the first rotation axis A1. In this manner, the contact portion 6464 of the tool member 6462 and the contact portion 6484 of the tool member 6482 can be actuated to engage or manipulate a target tissue during a surgical procedure.

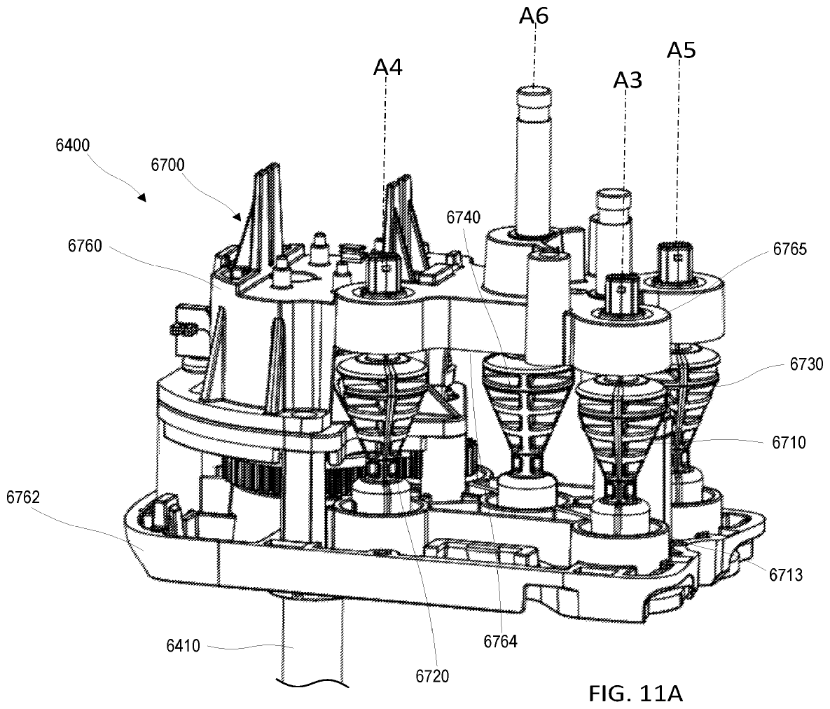

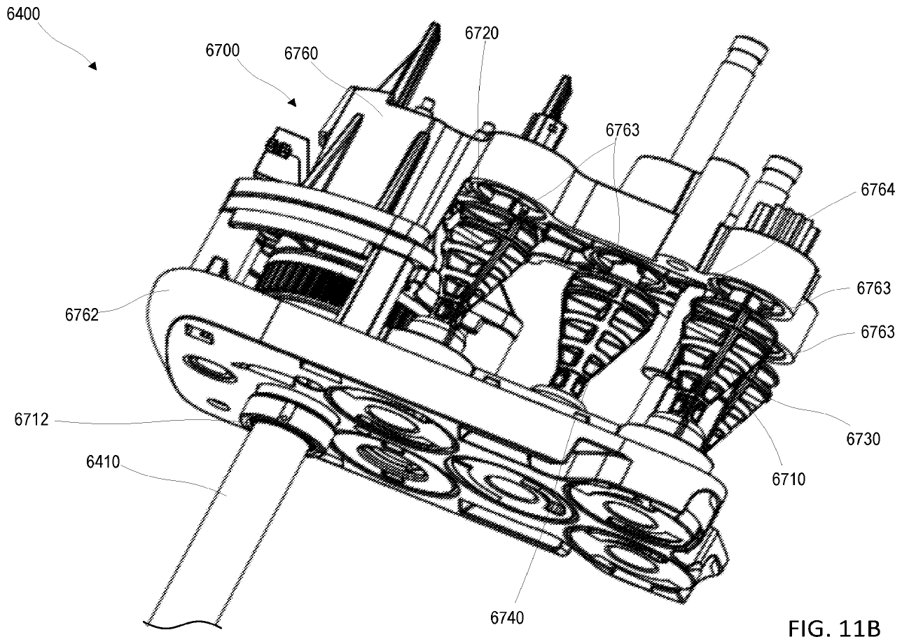

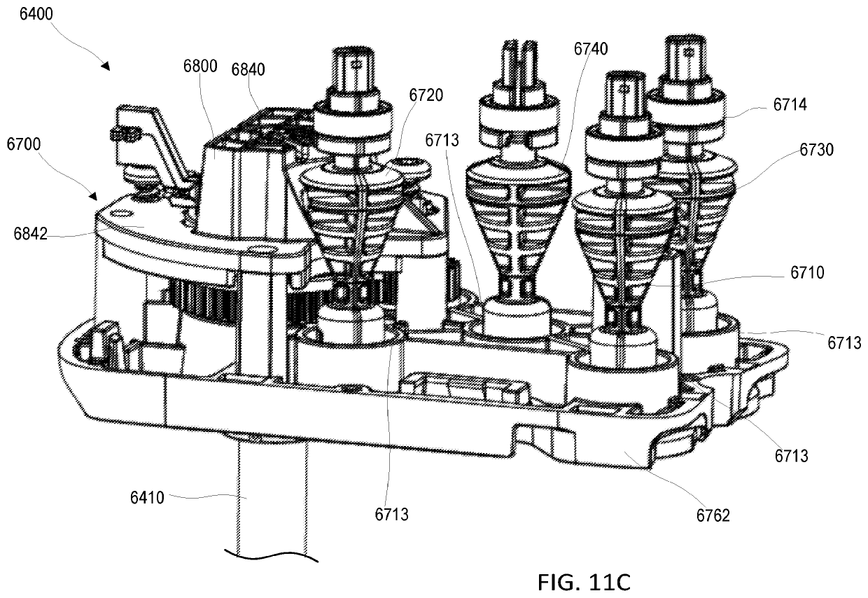

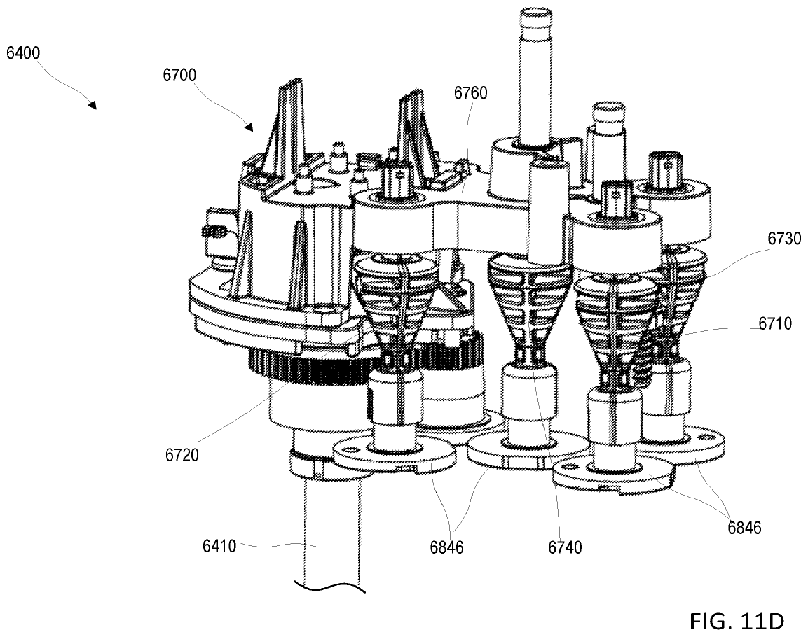

As shown in FIGS. 11A-11D, an example of the mechanical structure 6700 includes an upper chassis 6760, a lower chassis 6762, four capstans 6710, 6720, 6730, 6740, and a cable guide 2800. In some embodiments, the upper chassis 6760 and the lower chassis 6762 may partially enclose or fully enclose other components of mechanical structure 6700. In some embodiments, a housing cover (not shown) encloses the mechanical structure 6700, including the upper chassis 6760 and the lower chassis 6762. The lower chassis 6762 and the upper chassis 6760 provide structural support for mounting and aligning components in the mechanical structure 6700. For example, the lower chassis 6762 includes a shaft opening 6712 (see FIGS. 8 and 11B), within which the proximal end 6411 of the shaft 6410 is mounted. The lower chassis 6762 further includes one or more bearing surfaces or openings 6713, within which the capstans 6710, 6720, 6730, and 6740 are mounted and rotatably supported. The upper chassis 6760 also includes openings 6763 in a bottom 6864, within which an upper portion of the capstans are mounted as described in more detail below. The openings 6763 of the upper chassis 6760 are axially aligned with the openings 6713 of the lower chassis 6762 to support the capstans. In addition to providing mounting support for the internal components of the mechanical structure 6700, the lower chassis 6762 can include external features (e.g., recesses, clips, etc.) that interface with a docking port of a drive device (not shown). The drive device can be, for example, a handheld system or a computer-assisted teleoperated system that can receive the instrument 6400 and manipulate the instrument 6400 to perform various surgical operations. The drive device can include one or more motors to drive capstans of the mechanical structure 6700. In other embodiments, the drive device can be an assembly that can receive and manipulate the instrument 6400 to perform various operations.

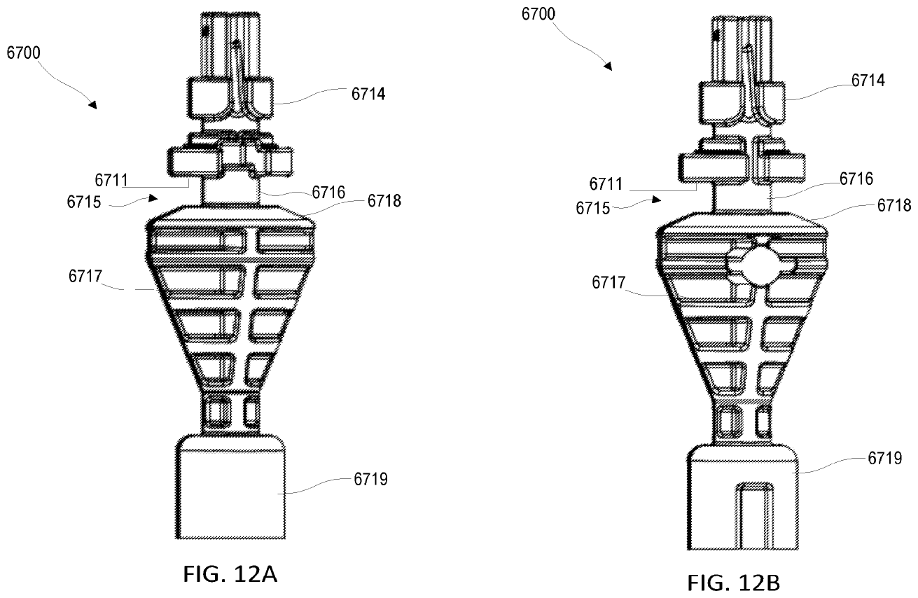

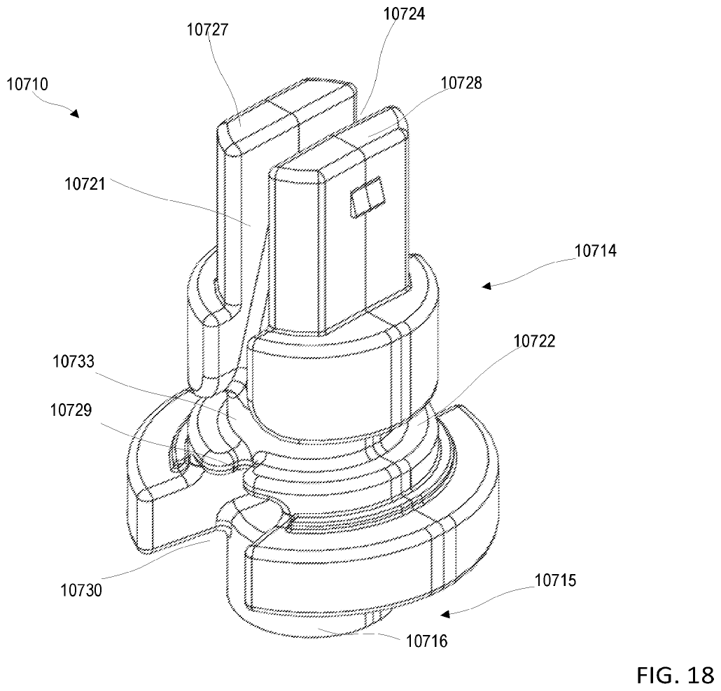

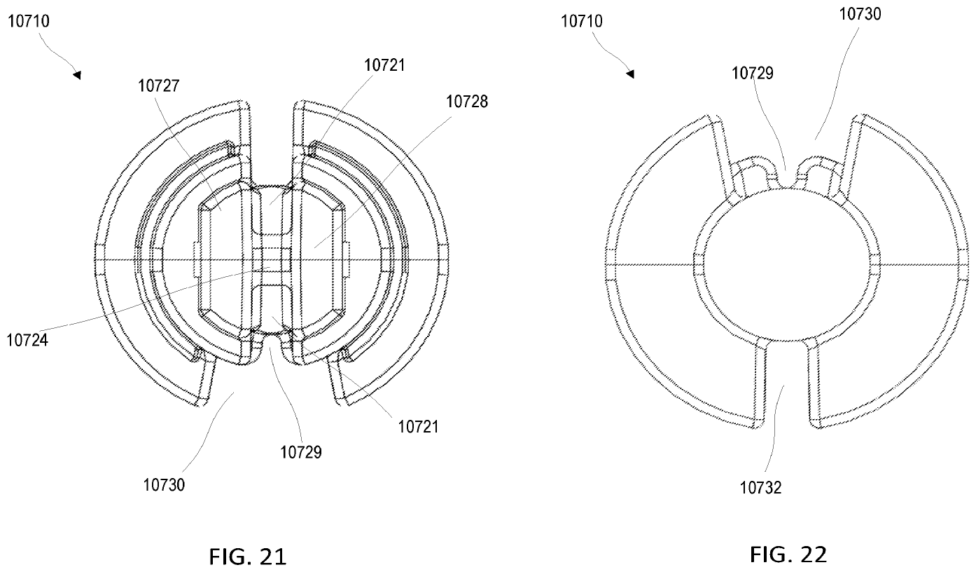

As shown in FIGS. 12A and 12B, the first capstan 6710 includes an upper portion 6714, a lower portion 6717 and a spool 6715 between the upper portion 6714 and the lower portion 6717. The upper portion 6714 functions as an anchor portion to secure the first cable 6420 to the capstan 6710. The upper portion 6714 can include a specific configuration to allow for a cable to be coupled to the capstan without the use of mechanical mechanisms (e.g., crimp joints, adhesive, knots) to maintain the coupling of the cable to the capstan 6710. Further details regarding coupling features of the upper portion 6714 and an example wrap sequence for coupling the cable 6420 to the upper portion 6714 of the first capstan 6710 are described below with reference to FIGS. 18-29. The spool 6715 includes a cable wrap surface 6716 (which functions as a drive surface) and a side wall 6718. The first cable 6420 is coupled to the first capstan 6710 such that a proximal end portion of the first cable 6420 wraps about the cable wrap surface 6716 of the first capstan 6710. In some embodiments, the cable 6420 wraps about the cable wrap surface 6716 no more than two revolutions. FIG. 14 shows the first cable 6420 coupled to the first capstan 6710 and the second capstan 6720, and the second cable 6420′ coupled to the third capstan 6730 and the fourth capstan 6740.

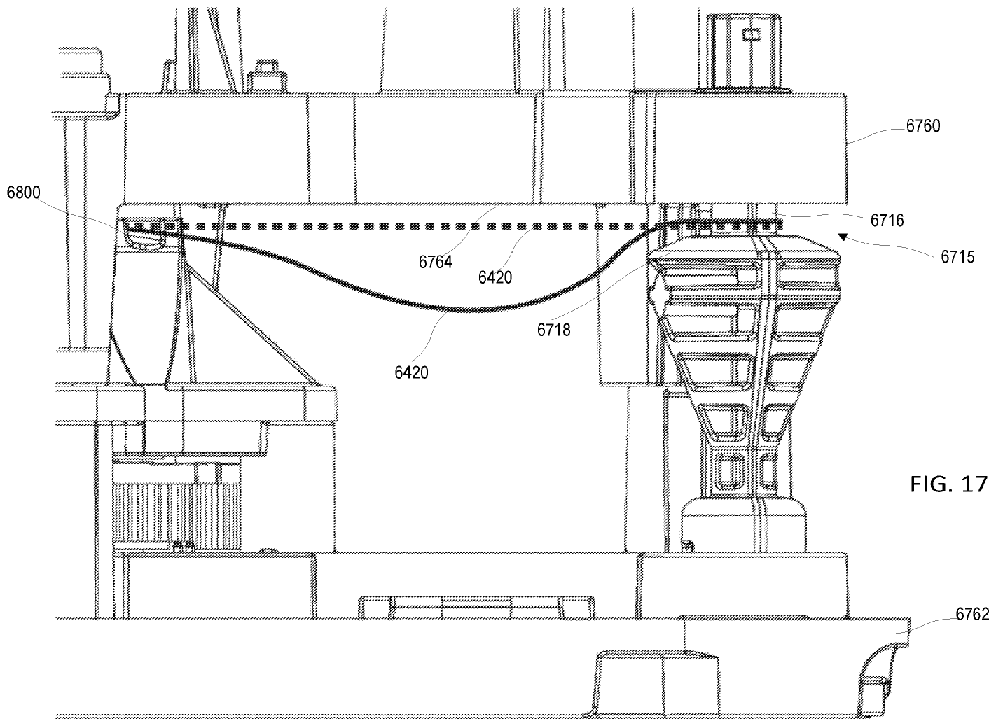

The lower portion 6717 of the first capstan 6710 is supported by the lower chassis 6762, and the upper portion 6714 of the first capstan 6710 is supported within the opening 6763 defined in the bottom 6764 of the upper chassis 6760 (see, e.g., FIG. 11B). In some embodiments, the bottom 6764 of the upper chassis 6760 has a continuous planar surface in which the openings 6763 are defined. In some embodiments, the bottom 6764 of the upper chassis has portions with surfaces in which the openings 6763 are defined, such as by the bottom of a support web structure of bracing material in the upper chassis. In some embodiments, a bottom 6711 of the upper portion 6714 (see, e.g., FIGS. 12A and 12B) of the first capstan 6710 is within the opening 6763 such that it is between the bottom 6764 of the upper chassis 6760 and a top surface 6765 of the upper chassis 6760. In other words, the entire upper portion 6714 of the first capstan 6710 is within the opening 6763. In some embodiments, the bottom 6711 of the upper portion 6714 of the first capstan 6710 is positioned flush with the bottom 6764 of the upper chassis 6760. The side wall 6718 of the spool 6715 slopes away from the bottom 6764 of the upper chassis 2760 (see, e.g., FIG. 17). In some embodiments, the side wall 6718 has a radiused outer edge surface to reduce friction and catch points between the cable 6420 and the capstan 6710 during operation of the medical device 6400. The second capstan 6720, third capstan 6730 and fourth capstan 6740 are each structured the same as the first capstan 6710 and can be supported by the lower chassis 6762 and the upper chassis 6760 in the same manner, and are therefore not described in detail here.

As described above, the upper portion 6714 of each of the capstans 6710, 6720, 6730, 6740 is rotatably supported within a corresponding opening 6763 of the upper chassis 6760. More specifically, a first bearing 6845 is coupled to the upper portion 6714 of the capstans and the first bearing 6845 is supported within the opening 6763. The first bearing 6845 is disposed over the portion of the cable 6420 that is coupled to the upper portion 6714 and thus assists in maintaining the coupling between the cable 6420 and the capstan 6710. The first bearing 6485 can be, for example, a rolling-element bearing, such as a ball or needle bearing.

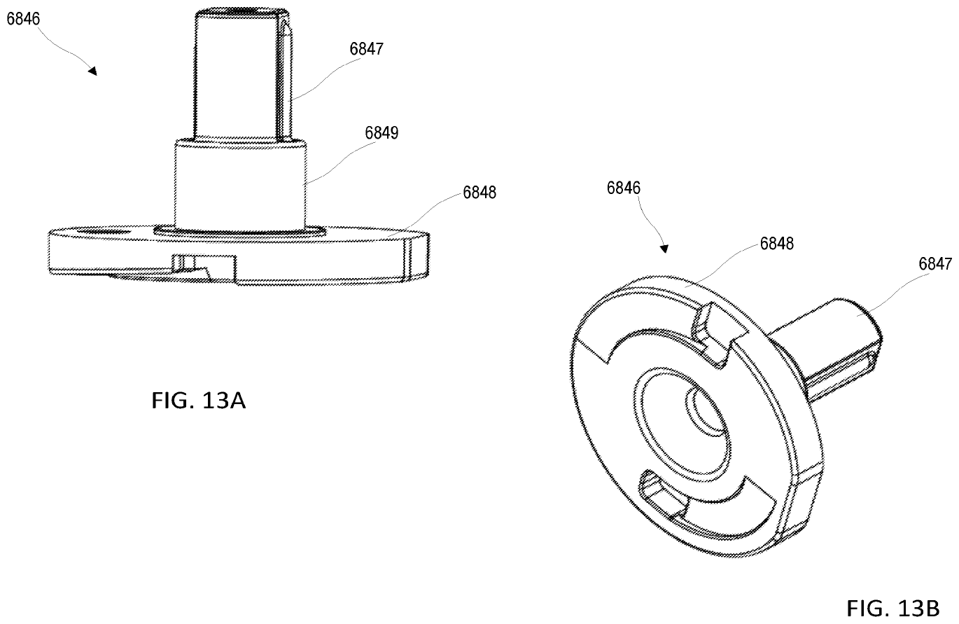

In addition, in this embodiment, the lower portions 6717 of each of the capstans 6710, 6720, 6730, 6740 is supported by the lower chassis 6762 via second bearings. In some embodiments, the drive discs 6846 can include a bearing surface 6849 that interface with journal bearings within the lower chassis 6762. As shown in FIGS. 13A and 13B, the drive discs 6846 include a neck 6847, a coupling portion 6848, and a bearing surface 6849. The neck 6846 is received within an opening (not shown) in the bottom end portion 6719 of the capstans 6710, 6720, 6730, and 6740. The coupling portion 6848 can be coupled to the lower chassis 6762, for example, within the openings 6713. As shown in FIG. 11B, the end surface of the drive portion 6848 is exposed and can be mated with a corresponding drive disc in a manipulator (not shown). Thus, motors can be operationally coupled to rotate the capstans via the drive discs 6846. The bearing surface 6849 interfaces with a journal bearing pressed within the lower chassis 6762. When the cable 6420 is in a tensioned state (e.g., operational state), the cable 6420 can cause a lateral load on the capstan 6720, and a portion of the lateral load on the first bearing 6845 is larger than a portion of the lateral load on the second bearing 6846. In some embodiments, the medical device 6400 may only include the first bearings 6845.

Each of capstans 6710, 6720, 6730, 6740 can be driven by a corresponding motor (not shown) in the drive device. For example, as shown in FIG. 11A, the first capstan 6710 can be driven to rotate about a first capstan axis A3, the second capstan 6720 can be driven to rotate about a second capstan axis A4, the third capstan 6730 can be driven to rotate about a third capstan axis A5, and the fourth capstan 6740 can be driven to rotate about a fourth capstan axis A6.

As shown in FIG. 14, a first proximal portion of the first cable 6420 is coupled to the first capstan 6710 of the mechanical structure 6700 and extends to a cable guide 6800 within the mechanical structure 6700 (see FIGS. 11C, 14, 15A and 15B), where it is rerouted through an interior passageway of the shaft 6410 (not shown in FIG. 14), and extends to the wrist assembly 6500 (not shown in FIG. 14), and to the end effector 6460 (not shown in FIG. 14). A distal portion of the first cable 6420 is coupled to the end effector 6460, and then the first cable 6420 extends proximally back through the interior passageway of the shaft 6410, proximally back through the cable guide 6800 and to the second capstan 6720, where a second proximal portion of the first cable 6420 is coupled to the second capstan 6720. Similarly, the second cable 6420′ is also routed between the mechanical structure 6700 and the end effector 6460. More specifically, the second cable 6420′ is coupled to the third capstan 6730 and extends to the cable guide 6800, where it is rerouted through the interior passageway of the shaft 6410, and the cable extends to the wrist assembly 6500 and to the end effector 6460. A distal portion of the second cable 6420′ is coupled to the end effector 6460, and then the second cable 6420′ extends back through the interior passageway of the shaft 6410, through the cable guide 6800, and to the fourth capstan 6740, where a second proximal portion of the second cable 6420′ is coupled to the fourth capstan 6740. Thus, the two proximal end portions of the cable 6420 are coupled to and actuated by two separate capstans (capstans 6710 and 6720) of the mechanical structure 6700. Likewise, the two proximal end portions of the second cable 6420′ are coupled to and actuated by two separate capstans (capstans 6730 and 6740). In alternative embodiments, the first cable 6420 and he second cable 6420′ can each be routed along an exterior of the shaft 6410 rather than within the interior passageway of the shaft 6410.

More specifically, the two ends of the first cable 6420 that are associated with opposing directions of a single degree of freedom are connected to two independent drive capstans 6710 and 6720, and the two ends of the second cable 6420′ that are associated with opposing directions of a single degree of freedom are connected to two independent drive capstans 6730 and 6740. This arrangement, which is generally referred to as an antagonist drive system (also described above with reference to FIGS. 7A-D), allows for independent control of the movement of (e.g., pulling in or paying out) each of the ends of the cables. The mechanical structure 6700 produces movement of the first cable 6420 and the second cable 6420′, which operates to produce the desired articulation movements (pitch, yaw, cutting or gripping) at the end effector 6460. Accordingly, as described herein, the mechanical structure 6700 includes components and controls to move a first portion of the first cable 6420 via the first capstan 6710 in a first direction (e.g., a proximal direction) and to move a second portion of the first cable 6420 via the second capstan 6720 in a second opposite direction (e.g., a distal direction). The mechanical structure 6700 can also move both the first portion of the first cable 6420 and the second portion of the first cable 6420 in the same direction. The mechanical structure 6700 also includes components and controls to move a first portion of the second cable 6420′ via the third capstan 6730 in a first direction (e.g., a proximal direction) and to move a second portion of the second cable via the fourth capstan 6740 in a second opposite direction (e.g., a distal direction). The mechanical structure 6700 can also move both the first portion of the second cable and the second portion of the second cable in the same direction. In this manner, the mechanical structure 6700 can maintain the desired tension within the cables to produce the desired movements at the end effector 6460.

As shown in FIGS. 15A and 15B, the cable guide 6800 includes an upper portion 6840 and a lower portion 6842. The lower portion 6842 is mounted to a component within the mechanical structure 6700, such as the lower chassis 6762. The upper portion 6840 includes multiple guide grooves 6831 on a top guide surface 6841. The guide grooves 6831 extend along the top guide surface 6841 to openings 6832 that are defined in the top surface 6841. As shown in FIG. 15B, the cables 6420, 6420 are routed along the top surface 6841 within the guide grooves 6831 and through the openings 6832 to be routed to the interior passageway of the shaft 6410. Each of the individual openings 6832 is in communication with a corresponding individual guide slot 6834, and each individual guide slot is defined by opposing protrusions 6833. The upper portion 6840 also includes an opening 6835 extending along a longitudinal length of the upper portion 6840. Opening 6835 is in communication with each individual guide slot 6834 to provide access for insertion of the cables during assembly of the medical device 6400.

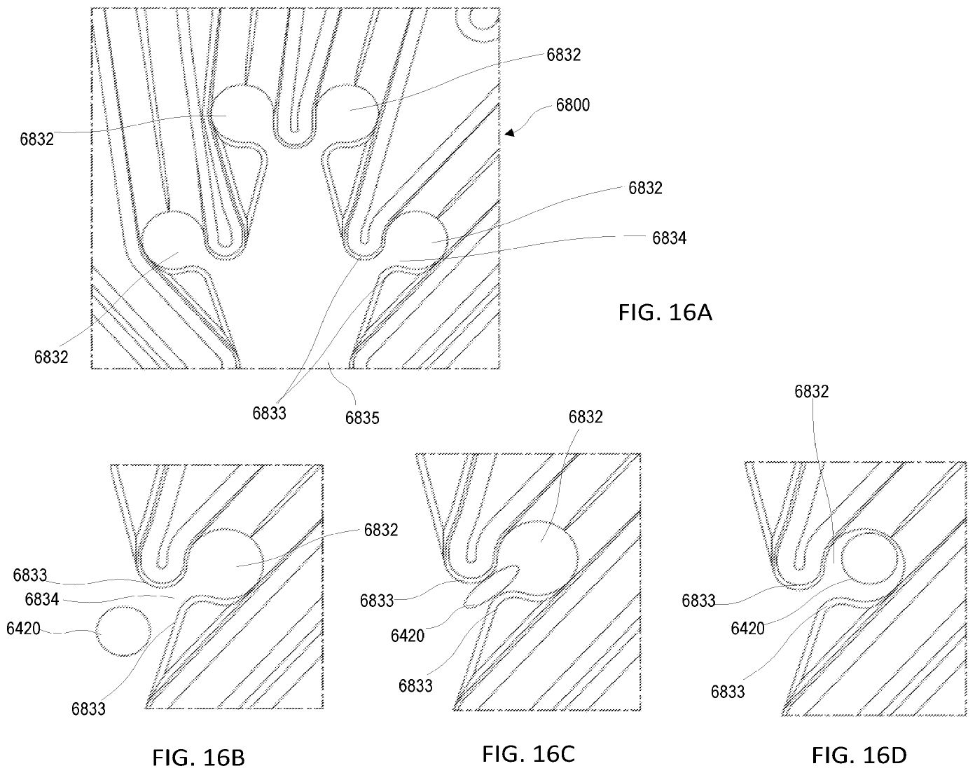

To insert the cables 6420, 6420′ into the openings 6832 during assembly of the medical device 6400, each individual portion of the cables 6420, 6420′ is passed from opening 6835, through the corresponding guide slot 6834 between the protrusions 6833, and then into the corresponding opening 6832. More specifically, FIGS. 16B-D illustrate an example insertion of the cable 6420 into the cable guide 6800. As shown in FIG. 16C, the cable 6420 is passed through the slot 6834 in a lateral direction such that a width of the cable 6420 compresses as it is passed through the slot 6832. For example, the cable 6420 can be formed with a braided polymer material such that the cross-sectional shape of cable 6420 can deform when an external force is applied to it. Thus, the cable 6420 can have a larger nominal width both under tension and when slack than the width of the guide slot 6834 and is able to compress to pass through the guide slot 6834 for insertion into opening 6432. After insertion, the cable 6420 can return back to its nominal width or diameter, or substantially the same as its nominal width or diameter, within the opening 6832 such that after insertion, the cable 6420 cannot be removed back through the guide slot 6834 without an external force exerted upon it (i.e., if there is slack it cannot pass through guide slot 6834). In some embodiments, the guide slot 6834 is, for example, 0.0254 cm (0.010 inches) at its smallest point, and the cable 6420 can have a nominal width or diameter, for example, of 0.0635 cm (0.025 inches).

As described herein and for medical devices 2400 and 3400, when the cable(s) (e.g., 6420 and cable 6420′) of the medical device 6400 are untensioned, for example, when the medical device 6400 is in storage, a slack loop of the cable will form within the mechanical structure 6700 between the capstans and a location between the capstans and proximal pf the shaft 6410. As shown in FIG. 17 for first cable 6420, a slack loop portion 6430 is shown between the first capstan 6710 and the cable guide 6800. Although only first cable 6420 and first capstan 6710 are shown in FIG. 17, it should be understood that a similar slack loop portion can be formed between the first cable 6460 and the second capstan 6720 and between the second cable 6420′ and the third and fourth capstans 6730 and 6740. In other embodiments, the slack loop portion 6430 of the cable 6420 can be, for example, between the capstans 6710 and 6720 and a location other than the cable guide 6800. FIG. 17 illustrates the first cable 6420 with a slack loop portion 6430 in the untensioned state and the first cable 6420 without a slack loop portion shown in dashed lines when in a tensioned state.

Rather than using mechanisms to eliminate or reduce the slack in the cable (e.g., a movable tensioner pulley, a spring-loaded guide surface that moves to maintain cable tension, or a ratchet mechanism to maintain cable tension), the mechanical structure 6700 includes structural features that can guide and control the slack loop portion 6430 to be maintained within a desired operational path within the mechanical structure 6700. More specifically, as shown in FIG. 17, when the cable 6420 is in an untensioned state (e.g., during storage), the slack loop portion 6430 exists in the cable 6420 between the cable guide 6800 and the spool 6715 of the capstan 6710. When the capstan 6710 rotates, the cable 6410 is wound about the cable wrap surface 6416 of the spool 6145, and the cable 6420 transitions from the untensioned state to the tensioned state as shown by the cable 6420 shown in dashed line in FIG. 17. During this transition, the slack loop portion 6430 of the cable 6420 is guided and maintained between the bottom 6764 of the upper chassis 6760 and the sloped side wall 6718 of the spool 6715 to ensure that the slack loop portion 6430 of the cable 6420 is properly routed to the cable guide 6800. As described above, the side wall of the spool 6715 has an outer circumference and a radiused edge surface at the outer circumference. The radiused outer edge surface can help reduce friction and catch points between the cable 6420 and the capstan 6710 during operation of the medical device.

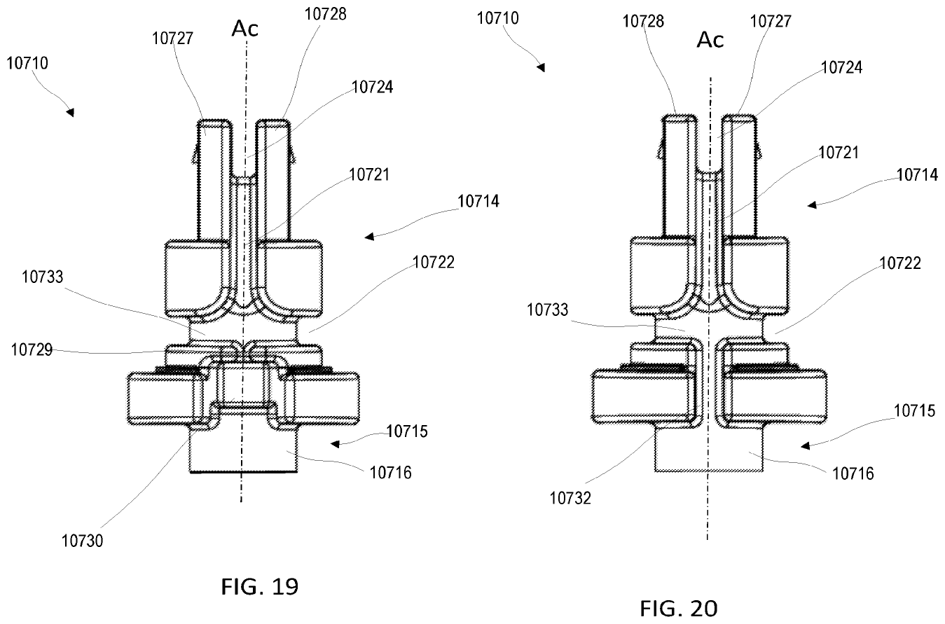

FIGS. 18-29 illustrate an upper portion 10714 of a capstan 10710 and a wrap sequence for coupling a cable 10420 to the capstan 10710. Only the upper portion 10714 of the capstan 10710 is shown for illustrative purposes. The capstan 10710 is structured the same as or similar to the capstans 6710, 6720, 6730 and 6740 described above and the wrap sequence described here for capstan 10710 can also be applied to capstans 6710, 6720, 6730 and 6740. As shown in FIGS. 18-22, the capstan portion 10710 includes a spool portion 10715 having a drive surface 10716, and the upper portion 10714 (which functions as an anchor portion to secure the cable to the capstan 10710). The upper portion 10714 has a coupling surface 10733. In this embodiment, the drive surface 10716 is a circular groove of the spool portion defined about a longitudinal axis Ac of the capstan 10710 (see e.g., FIGS. 19 and 20).

The upper portion 10714 of the capstan 10710 is cylindrical about the longitudinal axis Ac. The upper portion 10714 also includes a first slot 10721 that extends parallel to the longitudinal axis Ac and a second slot 10722 that crosses (is transverse to) the first slot 10721. In some embodiments, the first slot 10721 is perpendicular to the second slot 10722. The upper portion 10714 also defines a top slot 10724 defined between two posts 10727 and 10728 and that crosses the first slot 10721. As shown in FIGS. 18 and 19, a guide opening 10729 and an access opening 10730 are each defined on a first or front side of the capstan 10710. The guide opening 10729 can be used as a locator guide when coupling the cable 10420 to the capstan 10710 as described below. In some embodiments, the guide opening 10729 is sized larger than the size (e.g., diameter) of the cable 10420 such that the cable 10420 can be placed within the guide opening 10729 without exertion of force or friction between the capstan 10710 and the cable 10420. In some embodiments, the guide opening 10729 can be sized (e.g., width) smaller than the size (e.g., diameter) of the cable, such that a pinch point is created between the capstan 10710 and the cable 10420 to capture a portion of the cable 10420. In some embodiments, the guide opening 10729 can be a tapered passageway. The access opening 10730 can be used to provide access for a cutting tool to cut the first cable 10420 after coupling the first cable 10420 to the capstan 10710 as described in more detail below. As shown in FIG. 20, an elongate slot 10732 is defined on a second or back side of the capstan 10710, which can be used to route the first cable 10420 to the drive surface 10716 as described in more detail below.

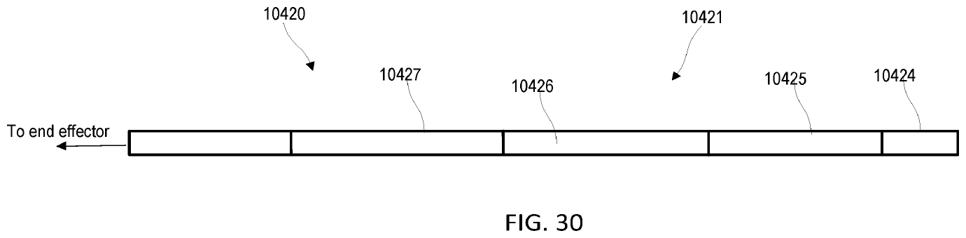

After being coupled to a tool member (e.g., 6462) of an end effector (e.g., 6460) in an example assembly operation, a proximal end portion 10421 of the cable 10420 (see, e.g., FIG. 30) extends along or through a shaft (e.g., 6410) and to the capstan 10710 of the mechanical structure in which the capstan 10710 is located to be coupled thereto. The proximal end portion 10421 of the first cable 10420 is routed along a particular path on the capstan 10710 and secured to the capstan without the need for a separate retention element (e.g., a crimp, retention member on the cable, or the like).

More specifically, the proximal end portion 10421 of the cable 10420 includes a termination end portion 10424, a first wrap portion 10425, a second wrap portion 10426, and a drive portion 10427, as shown in FIG. 30.

As shown in FIG. 23, the proximal end portion 10421 of cable 10420 extends from the end effector of the medical device and is placed within the guide opening 10729 such that the termination end portion 10424 of the first cable 10420 extends through the access opening 10730. At this point, there is a sufficient length of the proximal end portion 10421 of the cable 10420 extending between the termination end portion 10424 and the end effector to be wrapped about the capstan 10710 such that the cable 10420 extends between the end effector and the capstan 10710. The guide opening 10729 assists in positioning the cable 10420 within the first slot 10721 during coupling of the cable 10420 to the capstan 10710. A portion of the proximal end portion 10421, including the first wrap portion 10425, is routed upward through the first slot 10721 and over the third slot 10724 (as indicated at arrow 1 in FIG. 23).

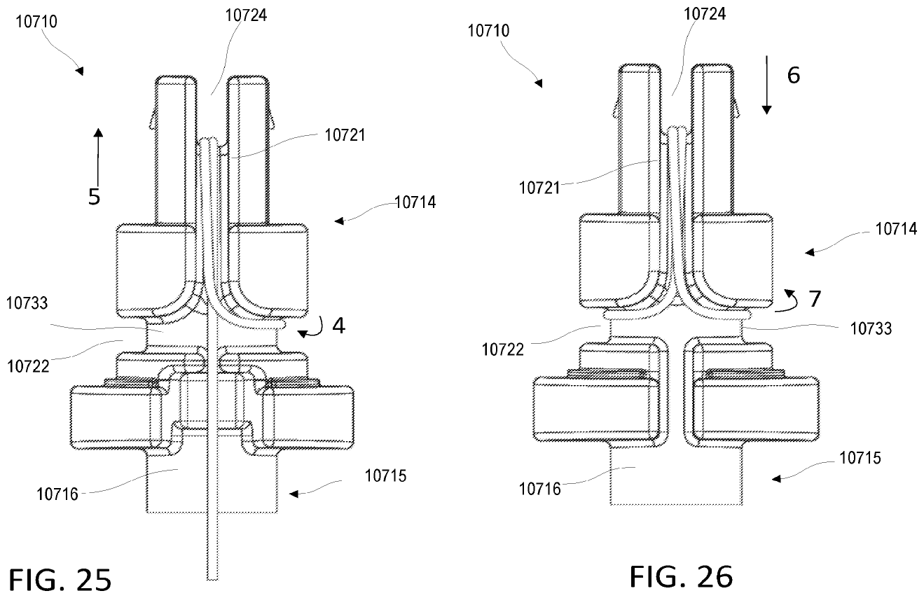

As shown in FIG. 24, a portion of the cable 10420 is then routed through the first slot 10721 on the second side of the capstan 10710 and is routed through the second slot 10722 and wrapped about the coupling surface 10733 towards the first side of the capstan 10710 as indicated at arrows 2 and 3 in FIG. 24. The portion of the cable 10420 is then routed within the second slot 10722 on the first side of the capstan 10710 and back up through the first slot 10721 as indicated at arrows 4 and 5 in FIG. 25.

As shown in FIG. 26, the portion of the cable 10420 is then routed through the first slot 10721 on the second side of the capstan 10710 and a second portion of the cable 10420, including the second wrap portion 10426, is routed through and wrapped about the coupling surface 10733 of the second slot 10722 in the opposite direction towards the first side of the capstan 10710, crossing over the first wrap portion 10425, as indicated at arrows 6 and 7 in FIG. 26.

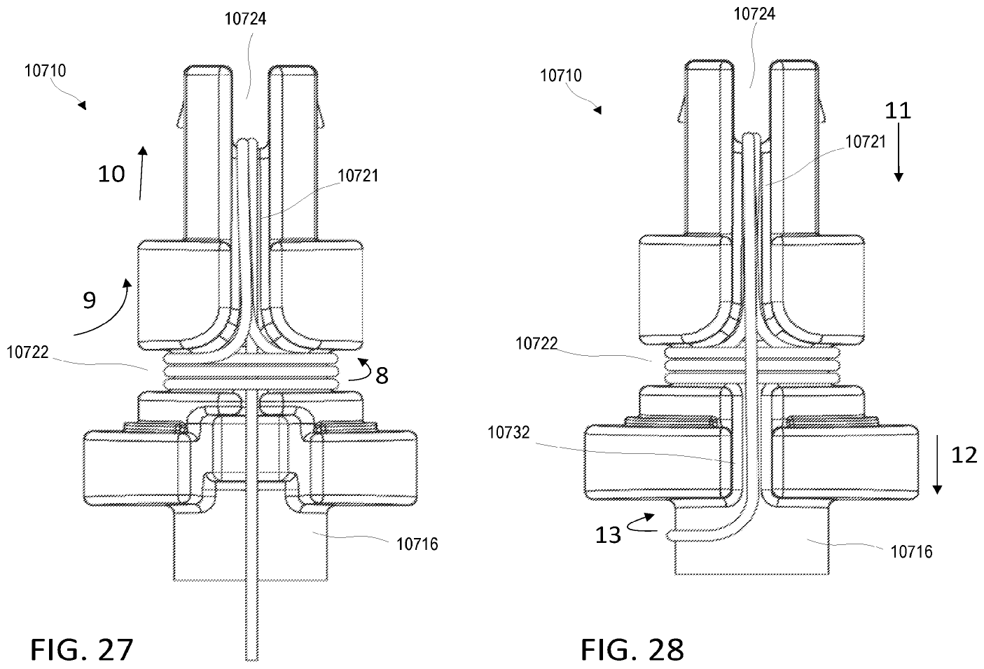

A portion of the cable 10420 is then wrapped about the coupling surface 10733 twice, as indicated at arrow 8 in FIG. 27, again crossing over the first wrap portion and crossing over the termination end portion 10424 of the cable 10420 that extends along the drive surface 10733 and within the guide opening 19729 and access opening 10732. As shown at arrows 9 and 10 in FIG. 27, a portion of the cable 10420 is then routed up through the slot 10721 and over the third slot 10724 again.

As shown in FIG. 28, a portion of the cable 10420 is then routed through the first slot 10721 on the second side of the capstan 10710, as shown at arrow 11, and is routed through the elongate slot 10732 as shown at arrow 12. A portion of the first cable 10420 is then wrapped back around the drive surface 10716 as indicated at arrow 13.

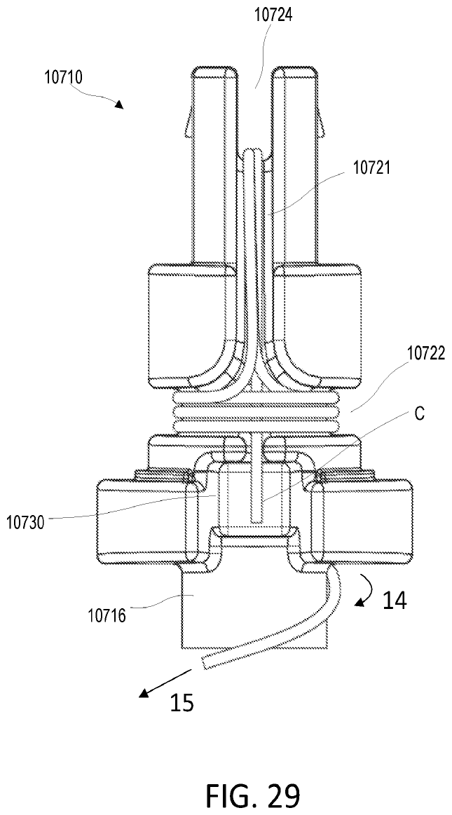

As shown in FIG. 29, a portion of the first cable 10420 is wrapped around the drive surface 10716 to the first side of the capstan 10710, as shown at arrow 14, and extends to the end effector 10460, as indicated at arrow 15.

After the first cable 10420 is coupled to the capstan 10710, the proximal end portion 10421 can be cut to remove excess cable. For example, as shown in FIG. 29, a cutting tool (not shown) can cut an end portion off the first cable 10420 at a location C within the access opening 10730. For example, the end can be cut with a heat cutter or by fusing the end or other suitable cutting tool. The length of the first cable 10420 is sized to enable the first cable 10420 to be coupled to the end effector 10460 and then coupled to the capstan 10710 such that there is slack in the cable during transport and storage. In other words, the cable 10420 is not in tension during transport and storage. By limiting the cable tension during storage, the amount of cable stretch can be reduced or eliminated.

With the first cable 10420 coupled to the proximal mechanical structure (not shown) and to the end effector 10460, rotational movement produced by the first capstan 10710 and the second capstan (not shown) can cause movement at the first tool member 10462 and the second tool member (not shown), respectively. Thus, as described previously, better control of the overall movement of the end effector 10460 (and tool members) can be achieved.

Although many of the embodiments described herein show a tool member (e.g., 10462) having a coupling spool that is separate from a drive pulley, in other embodiments, any of the tool members described herein can include a coupling portion (e.g., where the cable is wrapped to couple the cable to the tool member) that is also within (or a part of) the drive pulley portion. In this manner, the tool geometry can be made simpler by eliminating a separate coupling spool. For example, in some embodiments, a wrap groove can be defined by the drive surface of a drive pulley. Such a groove can be linear, or can be curved, or can have a zig-zag or switchback pattern. This construction can increase contact surface between the coupling portion and the cable to improve retention of the cable by the tool member.

While various embodiments have been described above, it should be understood that they have been presented by way of example only, and not limitation. Where methods and/or schematics described above indicate certain events and/or flow patterns occurring in certain order, the ordering of certain events and/or operations may be modified. While the embodiments have been particularly shown and described, it will be understood that various changes in form and details may be made.

For example, any of the instruments described herein (and the components therein) are optionally parts of a surgical assembly that performs minimally invasive surgical procedures, and which can include a manipulator unit, a series of kinematic linkages, a series of cannulas, or the like. Thus, any of the instruments described herein can be used in any suitable surgical system, such as the MIRS system 1000 shown and described above. Moreover, any of the instruments shown and described herein can be used to manipulate target tissue during a surgical procedure. Such target tissue can be cancer cells, tumor cells, lesions, vascular occlusions, thrombosis, calculi, uterine fibroids, bone metastases, adenomyosis, or any other bodily tissue. The presented examples of target tissue are not an exhaustive list. Moreover, a target structure can also include an artificial substance (or non-tissue) within or associated with a body, such as for example, a stent, a portion of an artificial tube, a fastener within the body or the like.

For example, any of the tool members can be constructed from any material, such as medical grade stainless steel, nickel alloys, titanium alloys or the like. Further, any of the links, tool members, tension members, or components described herein can be constructed from multiple pieces that are later joined together. For example, in some embodiments, a link can be constructed by joining together separately constructed components. In other embodiments however, any of the links, tool members, tension members, or components described herein can be monolithically constructed.

Although the instruments are generally shown as having an axis of rotation of the tool members (e.g., axis A1) that is normal to an axis of rotation of the wrist member (e.g., axis A2), in other embodiments any of the instruments described herein can include a tool member axis of rotation that is offset from the axis of rotation of the wrist assembly by any suitable angle.

Although various embodiments have been described as having particular features and/or combinations of components, other embodiments are possible having a combination of any features and/or components from any of embodiments as discussed above. Aspects have been described in the general context of medical devices, and more specifically surgical instruments, but inventive aspects are not necessarily limited to use in medical devices.

1. A medical device, comprising:
a chassis component comprising a bottom and an opening defined in the bottom; and
a capstan comprising an upper portion, a lower portion, and a spool between the upper portion and the lower portion;
wherein the upper portion of the capstan is supported within the opening of the chassis component; and
wherein the spool comprises a cable wrap surface and a side wall opposing the bottom of the chassis component.

2. The medical device of claim 1, wherein:
the medical device further comprises a cable guide and a cable;
the cable extends from the cable guide to the cable wrap surface of the spool; and
on a condition in which the cable is in an untensioned state, a slack loop exists in the cable between the cable guide and the spool.

3. The medical device of claim 2, wherein:
on a condition in which the capstan rotates to wind the cable about the cable wrap surface of the spool to cause the cable to transition from the untensioned state to a tensioned state, the slack loop of the cable is guided onto the spool by the bottom of the chassis component and the side wall of the spool.

4. (canceled)

5. The medical device of claim 1, wherein the side wall of the spool slopes away from the bottom of the chassis component.

6. The medical device of claim 1, wherein:
the chassis component comprises an upper chassis and a lower chassis, the opening of the chassis component is in the upper chassis;
the upper portion of the capstan is supported within the opening of the upper chassis; and
the lower portion of the capstan is supported by the lower chassis.

7. The medical device of claim 2, wherein:
the medical device further comprises a tool member;
the tool member moves from a first motion limit position through a defined range of motion to a second motion limit position; and
the cable is coupled to the spool such that the cable is routed about the cable wrap surface of the spool by no more than two revolutions as the spool rotates to move the tool member through the defined range of motion.

8. (canceled)

9. The medical device of claim 1, wherein:
the medical device further comprises a cable and a bearing;
the cable comprises a proximal portion coupled to the capstan; and
the bearing supports the upper portion of the capstan within the chassis component and surrounds the proximal portion of the cable.

10. The medical device of claim 9, wherein:
the proximal portion of the cable is coupled to the capstan in a wrapped configuration; and
the bearing surrounds the proximal portion of the cable to assist in maintaining the wrapped configuration of the proximal portion of the cable.

11. The medical device of claim 9, wherein the bearing is a first bearing:
the medical device further comprises a second bearing;
wherein the first bearing is coupled to the upper portion of the capstan; and
the second bearing supports the lower portion of the capstan.

12. (canceled)

13. The medical device of claim 11, wherein:
the spool of the capstan is located adjacent to the chassis component such that on a condition in which tension in the cable causes a lateral load on the capstan, a portion of the lateral load on the first bearing is larger than a portion of the lateral load on the second bearing.

14-15. (canceled)

16. The medical device of claim 1, wherein:
a diameter of the capstan tapers inward from the spool toward the lower portion of the capstan.

17. The medical device of claim 1, wherein:
the capstan comprises a plurality of radial ribs between the spool and the lower portion of the capstan; and
a diameter of each rib from the plurality of radial ribs decreases between the spool and the lower portion of the capstan.

18. (canceled)

19. The medical device of claim 2, wherein:
the upper portion of the capstan comprises a cable anchor portion; and
a proximal portion of the cable is secured to the capstan with a wrapping about the cable anchor portion.

20. A medical device, comprising:
a shaft comprising a distal end portion and a proximal end portion;
a tool member coupled to the distal end portion of the shaft;
a mechanical structure coupled to the proximal end portion of the shaft;
wherein the mechanical structure comprises a cable guide, a chassis component, and a capstan;
wherein the capstan comprises a first portion, a second portion, and a drive surface between the first portion and the second portion;
wherein the chassis component has a bottom; and
a cable coupled to the tool member, routed through the cable guide, and about the drive surface of the capstan;
wherein the cable comprises a slack loop between the drive surface of the capstan and the cable guide when the cable is in an untensioned state; and
wherein when the cable transitions from the untensioned state to a tensioned state, the slack loop of the cable is guided onto the capstan by the bottom of the chassis component and the second portion of the capstan.

21. The medical device of claim 20, wherein:
the cable is routed about the drive surface of the capstan by no more than two revolutions.

22. (canceled)

23. The medical device of claim 20, wherein:
the mechanical structure comprises a bearing;
the cable comprises a proximal portion coupled to the first portion of the capstan; and
wherein the bearing is coupled to the capstan and surrounds the proximal portion of the cable to assist in maintaining the proximal portion of the cable coupled to the first portion of the capstan.

24. The medical device of claim 23, wherein:
the bearing is a first bearing;
the medical device comprises a second bearing;
wherein the first bearing is coupled to the first portion of the capstan; and
the second bearing is coupled to the second portion of the capstan.

25. (canceled)

26. A medical device, comprising:
an instrument shaft, a cable guide, a capstan, and a cable;
wherein the instrument shaft comprises a shaft passageway;
wherein an opening is defined through the cable guide;
wherein the cable guide comprises two opposing protrusions, and the protrusions define a guide slot in communication with the opening of the cable guide;
wherein the cable has a first width;
wherein the guide slot has a second width smaller than the first width of the cable;
wherein the cable is routed from the capstan, through the opening of the cable guide, and into the shaft passageway; and
wherein on a condition in which the cable is in an untensioned state, the protrusions of the cable guide maintain the cable within the opening of the cable guide.

27. The medical device of claim 26, wherein:
the cable is coupled to the capstan such that the cable has a slack loop between the capstan and the cable guide when the cable is in the untensioned state.

28. The medical device of claim 27, wherein:
the medical device comprises a chassis component having a bottom; and
the capstan comprises a first portion, a second portion, and a drive surface;
wherein when the cable transitions from the untensioned state to a tensioned state, the slack loop of the cable is guided onto the capstan by the bottom of the chassis component and the second portion of the capstan.

29. (canceled)
